(19) United States
(12) Patent Application Publication
(10) Pub. No.: US 2023/0181563 A1
(43) Pub. Date: Jun. 15, 2023

(54) AKT3 MODULATORS AND METHODS OF USE THEREOF

(71) Applicant: Georgiamune LLC, Gaithersburg, MD (US)

(72) Inventors: Samir KHLEIF, Bethesda, MD (US); Mikayel MKRTICHYAN, Tujunga, CA (US)

(21) Appl. No.: 17/923,696

(22) PCT Filed: May 7, 2021
(86) PCT No.: PCT/US2021/031372

§ 371 Date: Nov. 7, 2022

Related U.S. Application Data
(60) Provisional application No. 63/021,797, filed on May 8, 2020.

(51) Int. Cl. A61K 31/4709; A61P 25/28; A61P 3/04; A61P 3/08; A61P 29/00; A61P 35/02

(52) U.S. Cl. CPC A61K 31/4709; A61P 25/28; A61P 3/04; A61P 3/08; A61P 29/00; A61P 35/02

(57) ABSTRACT

Compositions and methods of modulating Akt3 are disclosed herein. Also disclosed are methods of their use to treat or prevent various diseases. Activators and inhibitors of Akt3 are disclosed for use in treating and preventing various diseases.

### CROSS-REFERENCE TO RELATED APPLICATIONS

This application claims the benefit and priority of U.S. Provisional Application No. 63/021,797, filed on May 8, 2020, which is incorporated herein by reference in its entirety.

### INCORPORATION BY REFERENCE

Any patent, patent publication, journal publication, or other document cited herein is expressly incorporated herein by reference in its entirety.

### FIELD OF THE INVENTION

This invention is generally related to methods for treating and preventing diseases by modulating Akt3 signaling.

### BACKGROUND OF THE INVENTION

Chronic illnesses and diseases are long-lasting conditions that require ongoing medical attention and typically negatively affect the patient's quality of life. Chronic diseases are a leading cause of disability and death in the U.S. Common chronic diseases include, but are not limited to, inflammatory disease, neurodegenerative disease, pathogenic infection, immunodeficiency disorder, weight loss disorder, hormone imbalance, tuberous sclerosis, retinitis pigmentosa, and congestive heart failure. It is estimated that roughly 6 in 10 adults in the U.S. have a chronic disease, with 4 in 10 having two or more chronic diseases. Chronic diseases are also a leading driver of the U.S.'s $3.3 trillion annual health care costs (see National Center for Chronic Disease Prevention and Health Promotion). These staggering statistics emphasize the need for new and improved treatments and prophylactic interventions for chronic illnesses and diseases.

Neurodegenerative diseases are incurable, debilitating conditions that are characterized by the progressive degeneration and death of nerve cells, also called neurons. Neurons are the building blocks of the nervous system and do not usually reproduce or replace themselves when they become damaged or die. The loss or dysfunction of neurons in patients with neurodegenerative disease can affect body movement and brain function. Common neurodegenerative diseases include, but are not limited, to Alzheimer's disease, amyotrophic lateral sclerosis (ALS), Huntington's disease, Parkinson's disease, multiple sclerosis, prion disease, motor neuron disease, spinocerebellar ataxia, and spinal muscular atrophy. The symptoms of advanced neurodegenerative diseases can be devastating, with patients losing their memory, control over their movements, and their personality. There are currently no cures for neurodegenerative diseases and treatments that focus on managing symptoms typically confer negative side effects to the patient that further deteriorate their quality of life.

A serious complication of chronic diseases such as neurodegenerative diseases is cachexia, or wasting syndrome. Cachexia is defined as weight loss greater than 5% of body weight in 12 months or less in the presence of chronic illness. Other symptoms of cachexia include muscle atrophy, fatigue, weakness, and, often, loss of appetite. The weight loss associated with cachexia is due to the loss of not only fat but also muscle mass. Patients with cachexia often lose weight even if they are still eating a normal diet. There are currently no effective treatments for cachexia, which contributes to a large number of chronic disease-related deaths.

There is a growing need for more effective and tolerable treatments and prophylactic interventions for chronic diseases and complications associated with chronic disease.

Therefore, it is an object of the present invention to provide methods of treating and preventing chronic disease.

It is also an object of the present invention to provide methods of treating and preventing complications of chronic disease, such as cachexia.

### SUMMARY OF THE INVENTION

Compounds and pharmaceutical compositions for selectively modulating Akt3 are disclosed herein. Methods of using the compounds to treat or prevent various diseases and disorders are also disclosed. Non-limiting examples of disease include inflammatory disease, neurodegenerative disease, pathogenic infection, immunodeficiency disorder, weight loss disorder, hormone imbalance, tuberous sclerosis, retinitis pigmentosa, congestive heart failure, and a combination thereof.

Akt3 is highly expressed in the brain and its dysregulation has been implicated in a number of neurodegenerative diseases. Therefore, the disclosed Akt3 modulators can be useful for treating neurodegenerative diseases.

Methods of treating neurodegenerative diseases in a subject in need thereof are disclosed. In one embodiment, the method includes administering to the subject an activator of Akt3 in an amount effective to activate Akt3 and neuroprotective signaling downstream of Akt3 in brain tissue. In another embodiment, the method includes administering to the subject an inhibitor of Akt3 in an amount effective to inhibit Akt3 and neuroinflammatory signaling downstream of Akt3 in brain tissue.

In some embodiments, the neurodegenerative disease that is treated is an acute neurodegenerative disease selected from the group consisting of epilepsy, transient ischemia of the spinal cord, or cerebral ischemia. The neurodegenerative disease can also be a chronic neurodegenerative disease selected from the group consisting of Huntington's disease, Alzheimer's disease, Parkinson's disease, multiple sclerosis, spinal muscular atrophy, or amyotrophic lateral sclerosis.

Akt3 is also highly expressed in adipose tissue and adipocytes. Akt3 signaling has been implicated in adipogenesis. Therefore, the disclosed Akt3 modulators can be useful for treating diseases and disorders characterized by extreme weight loss.

Methods of treating extreme weight loss associated with conditions such as cachexia and anorexia are disclosed. The methods typically include administering to the subject an inhibitor of Akt3 in an amount effective to inhibit Akt3 signaling and promote adipogenesis in adipocytes.

In one embodiment, the subject has extreme weight loss due to cachexia. The cachexia can be associated with chronic diseases such as acquired immunodeficiency syndrome (AIDS), celiac disease, chronic obstructive pulmonary disease, multiple sclerosis, rheumatoid arthritis, congestive heart failure, tuberculosis, familial amyloid polyneuropathy, Crohn's disease, untreated and severe type 1 diabetes, anorexia nervosa, hyperthyroidism, and hormonal deficiency.

The disclosed methods can also include administering a second therapeutic agent to the subject. For neurodegenerative diseases, the second therapeutic agent can be an antispasmodic, a muscle relaxant, a pain reliever, an antidepressant, an antipsychotic, an anticonvulsant, an anticholinergic, or an anxiolytic. For subjects with extreme weight loss, the second therapeutic agent can be an appetite stimulant, nutrient supplementation, 5-HT3 antagonist, or Cox-2 inhibitor.

In one aspect, a method of treating a disease in a subject in need thereof is described, including administering to the subject a composition comprising an Akt3 modulator in an amount effective to modulate Akt3 signaling and treat or delay the progression of the disease.

In any one of the embodiments described herein, the disease is selected from the group consisting of neurodegenerative disease, cachexia, anorexia, obesity's complication, inflammatory disease, viral-induced inflammatory reaction, Gulf War Syndrome, tuberous sclerosis, retinitis pigmentosa, transplant rejection, cancer, ischemic tissue injury, traumatic tissue injury, and a combination thereof.

In any one of the embodiments described herein, the disease is the neurodegenerative disease.

In any one of the embodiments described herein, the neurodegenerative disease is selected from the group consisting of Parkinson's disease, Alzheimer's disease, amyotrophic lateral sclerosis, Motor Neuron Disease, Huntington's disease, HIV-induced neurodegeneration, Lewy Body Disease, spinal muscular atrophy, prion disease, spinocerebellar ataxia, familial amyloid polyneuropathy, and a combination thereof.

In any one of the embodiments described herein, the disease is cachexia or anorexia.

In any one of the embodiments described herein, the disease is obesity's complication.

In any one of the embodiments described herein, the obesity's complication is selected from the group consisting of glucose intolerance, hepatic steatosis, dyslipidemia, and a combination thereof.

In any one of the embodiments described herein, the disease is inflammatory disease.

In any one of the embodiments described herein, the inflammatory disease is selected from the group consisting of atopic dermatitis, allergy, asthma, and a combination thereof.

In any one of the embodiments described herein, the disease is viral-induced inflammatory reaction.

In any one of the embodiments described herein, the viral-induced inflammatory reaction is SARS-induced inflammatory pneumonitis, coronavirus disease 2019, or a combination thereof.

In any one of the embodiments described herein, the disease is Gulf War Syndrome or tuberous sclerosis.

In any one of the embodiments described herein, the disease is retinitis pigmentosa or transplant rejection.

In any one of the embodiments described herein, the disease is ischemic tissue injury or traumatic tissue injury.

In any one of the embodiments described herein, the disease is cancer.

In any one of the embodiments described herein, the cancer is selected from the group consisting of adult T-cell leukemia/lymphoma, bladder, brain, breast, cervical, colorectal, esophageal, kidney, liver, lung, nasopharyngeal, pancreatic, prostate, skin, stomach, uterine, ovarian, and testicular cancer.

In any one of the embodiments described herein, the cancer is leukemia.

In any one of the embodiments described herein, the leukemia is adult T-cell leukemia/lymphoma.

In any one of the embodiments described herein, the adult T-cell leukemia/lymphoma is caused by human T-cell lymphotropic virus.

In any one of the embodiments described herein, Akt3 is modulated in immune cells.

In any one of the embodiments described herein, the immune cells are selected from the group consisting of T cells, B cells, macrophages, and glial cells.

In any one of the embodiments described herein, the glial cells are astrocytes, microglia, or oligodendrocytes.

In any one of the embodiments described herein, the T cells are T regulatory cells.

In any one of the embodiments described herein, the Akt3 modulator activates Akt3 signaling.

In any one of the embodiments described herein, the Akt3 modulator inhibits Akt3 signaling.

In any one of the embodiments described herein, the Akt3 modulator increases T regulatory cell activity or production.

In any one of the embodiments described herein, the Akt3 modulator decreases T regulatory cell activity or production.

In any one of the embodiments described herein, the modulator of Akt3 is a compound according to Formula I:

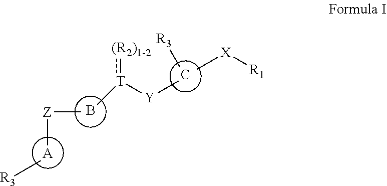

or a pharmaceutically acceptable enantiomer, salt, or solvate thereof, wherein:

rings A, B, and C are independently six-membered aryl or N-containing heteroaryl mono- or bicyclic ring systems containing zero or more N-atoms selected from the group consisting of phenyl, pyridine, pyrimidine, pyridazine, pyrazine, triazine, quinoline, quinazoline, isoquinoline, naphthalene, naphthyridine, indole, isoindole, cinnoline, phthalazine, quinoxaline, pteridine, purine, and benzimidazole;

R₁ is —(C₁-C₃₀)-alkyl, —(C₃-C₁₂)-cycloalkyl, —(C₃-C₁₂)-heterocycloalkyl, —(C₆-C₂₀)-aryl, or —(C₃-C₂₀)-heteroaryl groups optionally substituted by one or more substituents selected from —(C₁-C₁₂)-alkyl, —(C₃-C₁₂)-cycloalkyl, —(C₃-C₁₂)-heterocycloalkyl, —O—(C₁-C₁₂)-alkyl, —O—(C₁-C₁₂)-alkyl-(C₆-C₂₀)-aryl, —O—(C₃-C₁₂)-cycloalkyl, —S—(C₁-C₁₂)-alkyl, —S—(C₃-C₁₂)-cycloalkyl, —COO—(C₁-C₁₂)-alkyl, —COO—(C₃-C₁₂)-cycloalkyl, —CONH—(C₁-C₁₂)-alkyl, —CONH—(C₃-C₁₂)-cycloalkyl, —CO—(C₁-C₁₂)-alkyl, —CO—(C₃-C₁₂)-cycloalkyl, —N—[(C₁-C₁₂)-alkyl]₂, —(C₆-C₂₀)-aryl, —(C₆-C₂₀)-aryl-(C₁-C₁₂)-alkyl, —(C₆-C₂₀)-aryl-O—(C₁-C₁₂)-alkyl, —(C₃-C₂₀)-heteroaryl, —(C₃-C₂₀)-heteroaryl-(C₁-C₁₂)-alkyl, —(C₃-C₂₀)-heteroaryl-O—(C₁-C₁₂)-alkyl, —COOH, —OH, —SH, —SO₃H, —CN, —NH₂, or a halogen;

X, Y, and Z are independently ═O, —NH, —S, —N—(C₁-C₃₀)-alkyl, or —(C₁-C₃₀)-aryl;

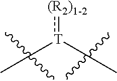

is selected from the group consisting of —CH((C₁-C₃₀)-alkyl))—, —(C═O)—, —CH(OH), —SO₂—, —SO—, and —CH(SOCH₃)—; and

R₃ is —(C₁-C₃₀)-alkyl, —(C₃-C₁₂)-cycloalkyl, —(C₃-C₁₂)-heterocycloalkyl, —(C₆-C₂₀)-aryl, or —(C₃-C₂₀)-heteroaryl groups optionally substituted by one or more substituents selected from —(C₁-C₁₂)-alkyl, —(C₃-C₁₂)-cycloalkyl, —(C₃-C₁₂)-heterocycloalkyl, —O—(C₁-C₁₂)-alkyl, —O—(C₁-C₁₂)-alkyl-(C₆-C₂₀)-aryl, —O—(C₃-C₁₂)-cycloalkyl, —S—(C₁-C₁₂)-alkyl, —S—(C₃-C₁₂)-cycloalkyl, —COO—(C₁-C₁₂)-alkyl, —COO—(C₃-C₁₂)-cycloalkyl, —CONH—(C₁-C₁₂)-alkyl, —CONH—(C₃-C₁₂)-cycloalkyl, —CO—(C₁-C₁₂)-alkyl, —CO—(C₃-C₁₂)-cycloalkyl, —N—[(C₁-C₁₂)-alkyl]₂, —(C₆-C₂₀)-aryl, —(C₆-C₂₀)-aryl-(C₁-C₁₂)-alkyl, —(C₆-C₂₀)-aryl-O—(C₁-C₁₂)-alkyl, —(C₃-C₂₀)-heteroaryl, —(C₃-C₂₀)-heteroaryl-(C₁-C₁₂)-alkyl, —(C₃-C₂₀)-heteroaryl-O—(C₁-C₁₂)-alkyl, —COOH, —OH, —SH, —SO₃H, —CN, —NH₂, or a halogen.

In any one of the embodiments described herein, the Akt3 modulator is a compound according to Formula II:

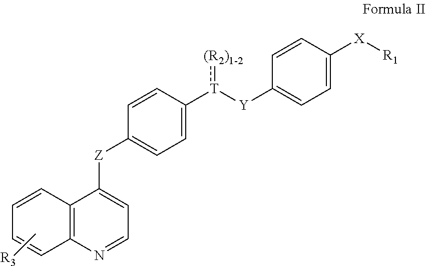

or a pharmaceutically acceptable enantiomer, salt, or solvate thereof, wherein:

R₁ is —(C₁-C₃₀)-alkyl, —(C₃-C₁₂)-cycloalkyl, —(C₃-C₁₂)-heterocycloalkyl, —(C₆-C₂₀)-aryl, or —(C₃-C₂₀)-heteroaryl groups optionally substituted by one or more substituents selected from —(C₁-C₁₂)-alkyl, —(C₃-C₁₂)-cycloalkyl, —(C₃-C₁₂)-heterocycloalkyl, —O—(C₁-C₁₂)-alkyl, —O—(C₁-C₁₂)-alkyl-(C₆-C₂₀)-aryl, —O—(C₃-C₁₂)-cycloalkyl, —S—(C₁-C₁₂)-alkyl, —S—(C₃-C₁₂)-cycloalkyl, —COO—(C₁-C₁₂)-alkyl, —COO—(C₃-C₁₂)-cycloalkyl, —CONH—(C₁-C₁₂)-alkyl, —CONH—(C₃-C₁₂)-cycloalkyl, —CO—(C₁-C₁₂)-alkyl, —CO—(C₃-C₁₂)-cycloalkyl, —N—[(C₁-C₁₂)-alkyl]₂, —(C₆-C₂₀)-aryl, —(C₆-C₂₀)-aryl-(C₁-C₁₂)-alkyl, —(C₆-C₂₀)-aryl-O—(C₁-C₁₂)-alkyl, —(C₃-C₂₀)-heteroaryl, —(C₃-C₂₀)-heteroaryl-(C₁-C₁₂)-alkyl, —(C₃-C₂₀)-heteroaryl-O—(C₁-C₁₂)-alkyl, —COOH, —OH, —SH, —SO₃H, —CN, —NH₂, or a halogen;

X, Y, and Z are independently —O, —NH, —S, —N—(C₁-C₃₀)-alkyl, or —(C₁-C₃₀)-aryl;

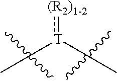

is selected from the group consisting of —CH((C₁-C₃₀)-alkyl))—, —(C═O)—, —CH(OH), —SO₂—, —SO—, and —CH(SOCH₃)—; and

R₃ is —(C₁-C₃₀)-alkyl, —(C₃-C₁₂)-cycloalkyl, —(C₃-C₁₂)-heterocycloalkyl, —(C₆-C₂₀)-aryl, or —(C₃-C₂₀)-heteroaryl groups optionally substituted by one or more substituents selected from —(C₁-C₁₂)-alkyl, —(C₃-C₁₂)-cycloalkyl, —(C₃-C₁₂)-heterocycloalkyl, —O—(C₁-C₁₂)-alkyl, —O—(C₁-C₁₂)-alkyl-(C₆-C₂₀)-aryl, —O—(C₃-C₁₂)-cycloalkyl, —S—(C₁-C₁₂)-alkyl, —S—(C₃-C₁₂)-cycloalkyl, —COO—(C₁-C₁₂)-alkyl, —COO—(C₃-C₁₂)-cycloalkyl, —CONH—(C₁-C₁₂)-alkyl, —CONH—(C₃-C₁₂)-cycloalkyl, —CO—(C₁-C₁₂)-alkyl, —CO—(C₃-C₁₂)-cycloalkyl, —N—[(C₁-C₁₂)-alkyl]₂, —(C₆-C₂₀)-aryl, —(C₆-C₂₀)-aryl-(C₁-C₁₂)-alkyl, —(C₆-C₂₀)-aryl-O—(C₁-C₁₂)-alkyl, —(C₃-C₂₀)-heteroaryl, —(C₃-C₂₀)-heteroaryl-(C₁-C₁₂)-alkyl, —(C₃-C₂₀)-heteroaryl-O—(C₁-C₁₂)-alkyl, —COOH, —OH, —SH, —SO₃H, —CN, —NH₂, or a halogen.

In any one of the embodiments described herein, the Akt3 modulator is a compound according to Formula III:

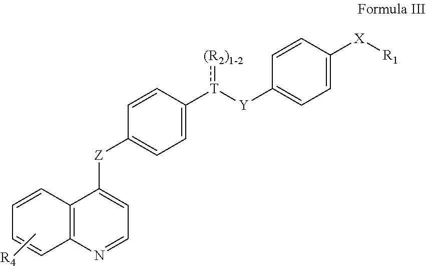

or a pharmaceutically acceptable enantiomer, salt, or solvate thereof, wherein:

R₁ is —(C₁-C₃₀)-alkyl, —(C₃-C₁₂)-cycloalkyl, —(C₃-C₁₂)-heterocycloalkyl, —(C₆-C₂₀)-aryl, or —(C₃-C₂₀)-heteroaryl groups optionally substituted by one or more substituents selected from —(C₁-C₁₂)-alkyl, —(C₃-C₁₂)-cycloalkyl, —(C₃-C₁₂)-heterocycloalkyl, —O—(C₁-C₁₂)-alkyl, —O—(C₁-C₁₂)-alkyl-(C₆-C₂₀)-aryl, —O—(C₃-C₁₂)-cycloalkyl, —S—(C₁-C₁₂)-alkyl, —S—(C₃-C₁₂)-cycloalkyl, —COO—(C₁-C₁₂)-alkyl, —COO—(C₃-C₁₂)-cycloalkyl, —CONH—(C₁-C₁₂)-alkyl, —CONH—(C₃-C₁₂)-cycloalkyl, —CO—(C₁-C₁₂)-alkyl, —CO—(C₃-C₁₂)-cycloalkyl, —N—[(C₁-C₁₂)-alkyl]₂, —(C₆-C₂₀)-aryl, —(C₆-C₂₀)-aryl-(C₁-C₁₂)-alkyl, —(C₆-C₂₀)-aryl-O—(C₁-C₁₂)-alkyl, —(C₃-C₂₀)-heteroaryl, —(C₃-C₂₀)-heteroaryl-(C₁-C₁₂)-alkyl, —(C₃-C₂₀)-heteroaryl-O—(C₁-C₁₂)-alkyl, —COOH, —OH, —SH, —SO₃H, —CN, —NH₂, or a halogen;

X, Y, and Z are independently —O, —NH, —S, —N—(C₁-C₃₀)-alkyl, or —(C₁-C₃₀)-aryl;

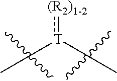

is —CH((C₁-C₃₀)-alkyl))—, —(C═O)—, —CH(OH), —SO₂—, —SO—, or —CH(SOCH₃)—; and

R₄ is —(C₁-C₁₂)-alkyl, —(C₃-C₁₂)-cycloalkyl, —(C₃-C₁₂)-heterocycloalkyl, —O—(C₁-C₁₂)-alkyl, —O—(C₁-C₁₂)-alkyl-(C₆-C₂₀)-aryl, —O—(C₃-C₁₂)-cycloalkyl, —S—(C₁-C₁₂)-alkyl, —S—(C₃-C₁₂)-cycloalkyl, —COO—(C₁-C₁₂)-alkyl, —COO—(C₃-C₁₂)-cycloalkyl, —CONH—(C₁-C₁₂)-alkyl, —CONH—(C₃-C₁₂)-cycloalkyl, —CO—(C₁-C₁₂)-alkyl, —CO—(C₃-C₁₂)-cycloalkyl, —N—[(C₁-C₁₂)-alkyl]₂, —(C₆-C₂₀)-aryl, —(C₆-C₂₀)-aryl-(C₁-C₁₂)-alkyl, —(C₆-C₂₀)-aryl-O—(C₁-C₁₂)-alkyl, —(C₃-C₂₀)-heteroaryl, —(C₃-C₂₀)-heteroaryl-(C₁-C₁₂)-alkyl, —(C₃-C₂₀)-heteroaryl-O—(C₁-C₁₂)-alkyl, —COOH, —OH, —SH, —SO₃H, —CN, —NH₂, or a halogen.

In any one of the embodiments described herein, the Akt3 modulator is a compound according to Formula IV:

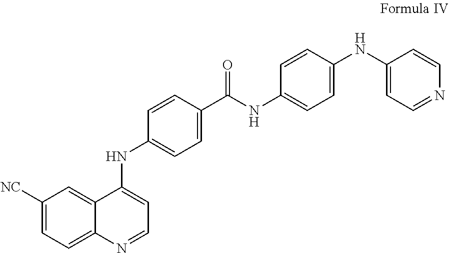 [SMILES: N#Cc1ccc2nccc(Nc3ccc(C(=O)Nc4ccc(Nc5ccncc5)cc4)cc3)c2c1]

or a pharmaceutically acceptable enantiomer, salt, or solvate thereof.

In any one of the embodiments described herein, the method further includes administering a second therapeutic agent to the subject.

In any one of the embodiments described herein, the second therapeutic agent is selected from the group consisting of a nutrient supplementation, a chemotherapeutic, an anti-inflammatory, an immunosuppressant, a cholinesterase inhibitor, an antidepressant, an anxiolytic, an antipsychotic, riluzole, edavarone, a dopamine agonist, a MAO B inhibitor, a catechol O-methyltransferase inhibitor, an anticholinergic, an anticonvulsant, tetrabenazine, carbidopa-levodopa, an antispastic, an antibody, a fusion protein, an enzyme, a nucleic acid, a ribonucleic acid, an anti-proliferative, a cytotoxic agent, an appetite stimulant, a 5-HT3 antagonist, a Cox-2 inhibitor, and a combination thereof.

In another aspect, a method of treating cachexia in a subject in need thereof is described, including administering a composition comprising a selective inhibitor of Akt3 to the subject in an amount effective to inhibit Akt3 signaling in adipocytes and activate adipogenesis.

In any one of the embodiments described herein, the method further includes administering a second therapeutic agent to the subject.

In any one of the embodiments described herein, the second therapeutic agent is selected from the group consisting of an appetite stimulant, a nutrient supplementation, a 5-HT3 antagonist, a Cox-2 inhibitor, a chemotherapeutic, an anti-inflammatory, an immunosuppressant, a cholinesterase inhibitor, an antidepressant, an anxiolytic, an antipsychotic, riluzole, edavarone, a dopamine agonist, a MAO B inhibitor, a catechol O-methyltransferase inhibitor, an anticholinergic, an anticonvulsant, tetrabenazine, carbidopa-levodopa, an antispastic, an antibody, a fusion protein, an enzyme, a nucleic acid, a ribonucleic acid, an anti-proliferative, a cytotoxic agent, and a combination thereof.

In any one of the embodiments described herein, the second therapeutic agent is an appetite stimulant, a nutrient supplementation, a 5-HT3 antagonist, or a Cox-2 inhibitor.

In any one of the embodiments described herein, the subject has neurodegenerative disease, cachexia, anorexia, obesity's complication, inflammatory disease, viral-induced inflammatory reaction, Gulf War Syndrome, tuberous sclerosis, retinitis pigmentosa, transplant rejection, cancer, and a combination thereof.

In any one of the embodiments described herein, the Akt3 inhibitor is a compound selected from the group consisting of:

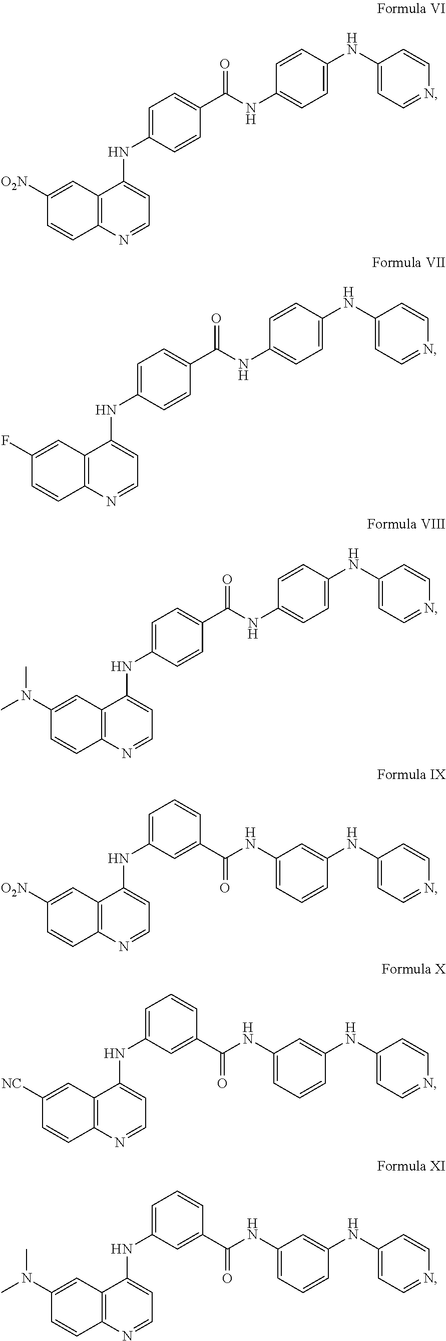 [SMILES: O=C(Nc1ccc(Nc2ccncc2)cc1)c1ccc(Nc2ccnc3ccc([N+](=O)[O-])cc23)cc1 ; O=C(Nc1ccc(Nc2ccncc2)cc1)c1ccc(Nc2ccnc3ccc(F)cc23)cc1 ; CN(C)c1ccc2nccc(Nc3ccc(C(=O)Nc4ccc(Nc5ccncc5)cc4)cc3)c2c1 ; O=C(Nc1cccc(Nc2ccncc2)c1)c1cccc(Nc2ccnc3ccc([N+](=O)[O-])cc23)c1 ; N#Cc1ccc2nccc(Nc3cccc(C(=O)Nc4cccc(Nc5ccncc5)c4)c3)c2c1 ; CN(C)c1ccc2nccc(Nc3cccc(C(=O)Nc4cccc(Nc5ccncc5)c4)c3)c2c1]

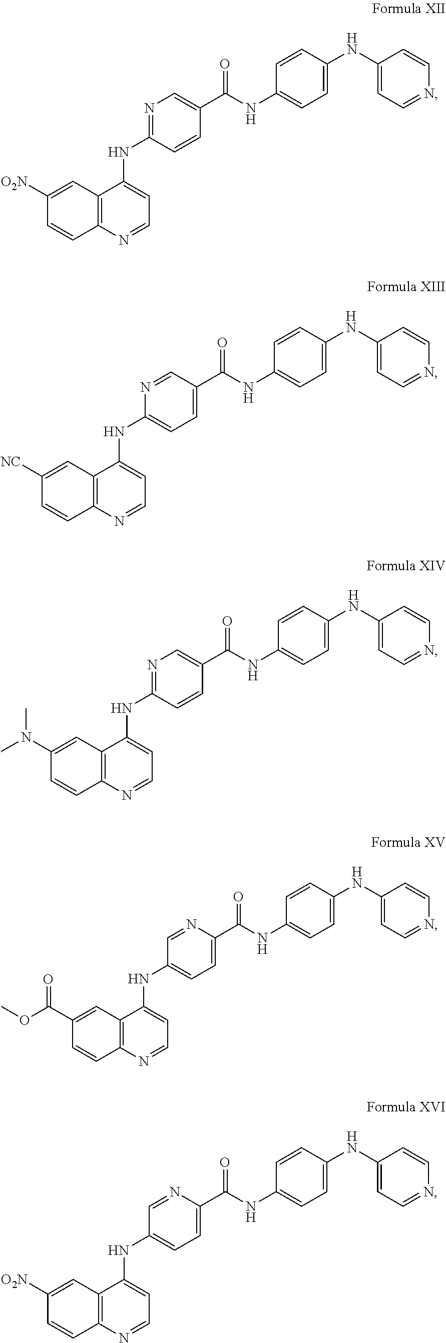 [SMILES: O=C(Nc1ccc(Nc2ccncc2)cc1)c1ccc(Nc2ccnc3ccc([N+](=O)[O-])cc23)nc1 ; N#Cc1ccc2nccc(Nc3ccc(C(=O)Nc4ccc(Nc5ccncc5)cc4)cn3)c2c1 ; CN(C)c1ccc2nccc(Nc3ccc(C(=O)Nc4ccc(Nc5ccncc5)cc4)cn3)c2c1 ; COC(=O)c1ccc2nccc(Nc3ccc(C(=O)Nc4ccc(Nc5ccncc5)cc4)nc3)c2c1 ; O=C(Nc1ccc(Nc2ccncc2)cc1)c1ccc(Nc2ccnc3ccc([N+](=O)[O-])cc23)cn1]

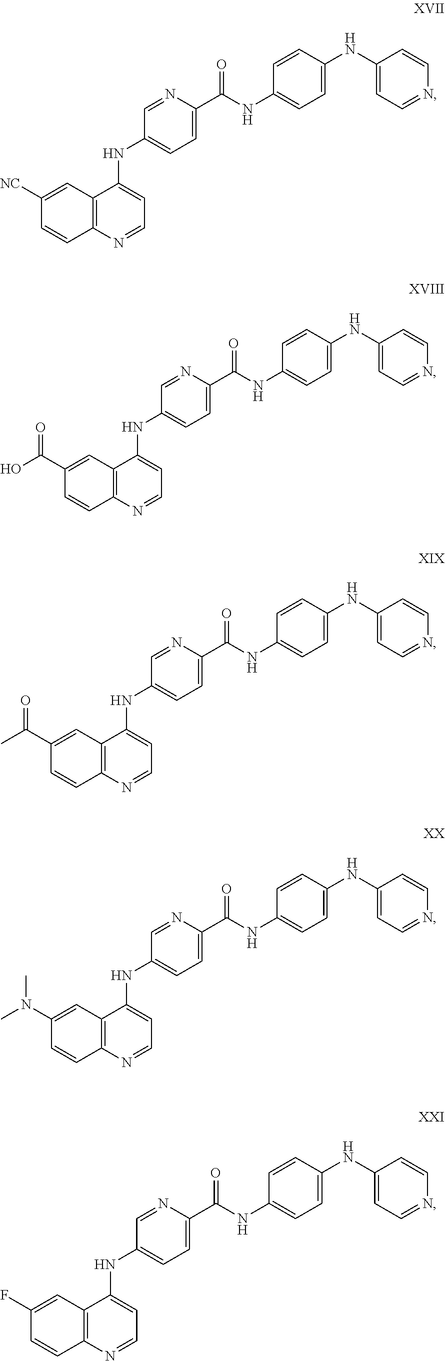 [SMILES: N#Cc1ccc2nccc(Nc3ccc(C(=O)Nc4ccc(Nc5ccncc5)cc4)nc3)c2c1 ; O=C(O)c1ccc2nccc(Nc3ccc(C(=O)Nc4ccc(Nc5ccncc5)cc4)nc3)c2c1 ; CC(=O)c1ccc2nccc(Nc3ccc(C(=O)Nc4ccc(Nc5ccncc5)cc4)nc3)c2c1 ; CN(C)c1ccc2nccc(Nc3ccc(C(=O)Nc4ccc(Nc5ccncc5)cc4)nc3)c2c1 ; O=C(Nc1ccc(Nc2ccncc2)cc1)c1ccc(Nc2ccnc3ccc(F)cc23)cn1]

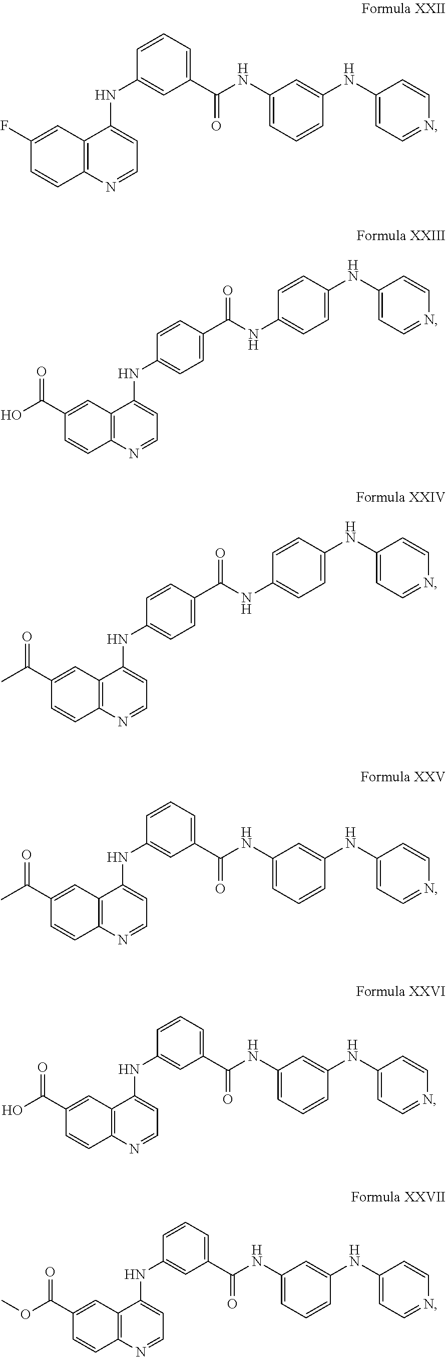 [SMILES: O=C(Nc1cccc(Nc2ccncc2)c1)c1cccc(Nc2ccnc3ccc(F)cc23)c1 ; O=C(O)c1ccc2nccc(Nc3ccc(C(=O)Nc4ccc(Nc5ccncc5)cc4)cc3)c2c1 ; CC(=O)c1ccc2nccc(Nc3ccc(C(=O)Nc4ccc(Nc5ccncc5)cc4)cc3)c2c1 ; CC(=O)c1ccc2nccc(Nc3cccc(C(=O)Nc4cccc(Nc5ccncc5)c4)c3)c2c1 ; O=C(O)c1ccc2nccc(Nc3cccc(C(=O)Nc4cccc(Nc5ccncc5)c4)c3)c2c1 ; COC(=O)c1ccc2nccc(Nc3cccc(C(=O)Nc4cccc(Nc5ccncc5)c4)c3)c2c1]

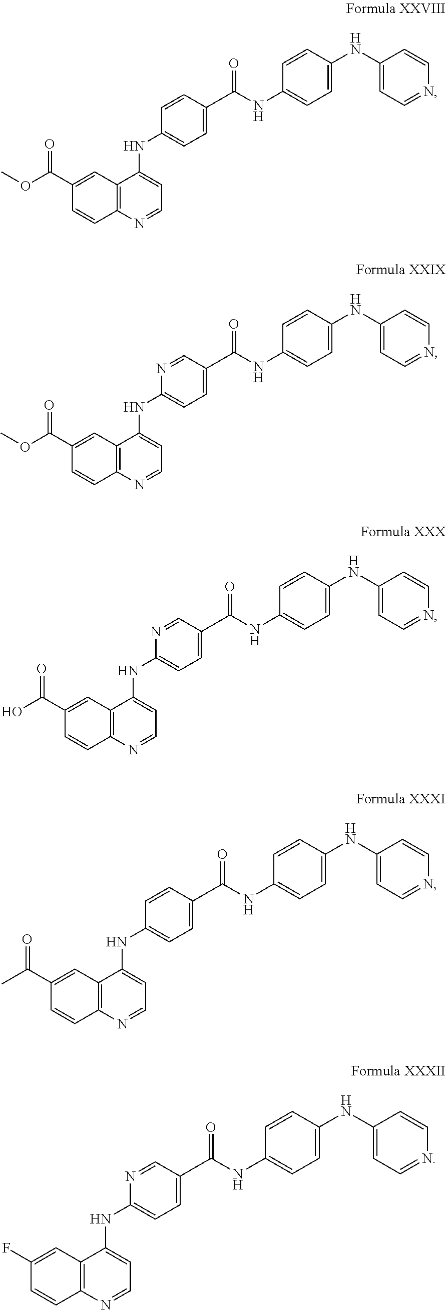 [SMILES: COC(=O)c1ccc2nccc(Nc3ccc(C(=O)Nc4ccc(Nc5ccncc5)cc4)cc3)c2c1 ; COC(=O)c1ccc2nccc(Nc3ccc(C(=O)Nc4ccc(Nc5ccncc5)cc4)cn3)c2c1 ; O=C(O)c1ccc2nccc(Nc3ccc(C(=O)Nc4ccc(Nc5ccncc5)cc4)cn3)c2c1 ; CC(=O)c1ccc2nccc(Nc3ccc(C(=O)Nc4ccc(Nc5ccncc5)cc4)cc3)c2c1 ; O=C(Nc1ccc(Nc2ccncc2)cc1)c1ccc(Nc2ccnc3ccc(F)cc23)nc1]

### DETAILED DESCRIPTION OF THE INVENTION

### I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any compositions, methods, and materials similar or equivalent to those described herein can be used in the practice or testing of the present invention. All publications mentioned herein are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approximately ±10%; in other embodiments the values may range in value either above or below the stated value in a range of approximately ±5%; in other embodiments the values may range in value either above or below the stated value in a range of approximately ±2%; in other embodiments the values may range in value either above or below the stated value in a range of approximately ±1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise indicated. No language in the specification should be construed as indicating any non-claimed element as essential to the practice of the invention.

As used herein, the terms “cancer” and, equivalently, “tumor” refer to a condition in which abnormally replicating cells of host origin are present in a detectable amount in a subject. The cancer can be a malignant or non-malignant cancer. Cancers or tumors include, but are not limited to, adult T-cell leukemia/lymphoma (including that caused by human T-cell lymphotropic virus (HTLV-1)), biliary tract cancer; brain cancer; breast cancer; cervical cancer; choriocarcinoma; colon cancer; endometrial cancer; esophageal cancer; gastric (stomach) cancer; intraepithelial neoplasms; leukemias; lymphomas; liver cancer; lung cancer (e.g., small cell and non-small cell); melanoma; neuroblastomas; oral cancer; ovarian cancer; pancreatic cancer; prostate cancer; rectal cancer; renal (kidney) cancer; sarcomas; skin cancer; testicular cancer; thyroid cancer; as well as other carcinomas and sarcomas. As used herein, the term “lymphoma” refers to cancer of the lymphatic system or a blood cancer that develops from lymphocytes. Cancers can be primary or metastatic. Diseases other than cancers may be associated with mutational alternation of component of Ras signaling pathways and the compound disclosed herein may be used to treat these non-cancer diseases. Such non-cancer diseases may include: neurofibromatosis; Leopard syndrome; Noonan syndrome; Legius syndrome; Costello syndrome; Cardio-facio-cutaneous syndrome; hereditary gingival fibromatosis type 1; autoimmune lymphoproliferative syndrome; and capillary malformation-arterovenous malformation.

The term “stimulate expression of” means to affect expression of, for example, to induce expression or activity, or induce increased/greater expression or activity relative to normal, healthy controls.

The terms “immune activating response”, “activating immune response”, and “immune stimulating response” refer to a response that initiates, induces, enhances, or increases the activation or efficiency of innate or adaptive immunity. Such immune responses include, for example, the development of a beneficial humoral (antibody-mediated) and/or a cellular (mediated by antigen-specific T cells or their secretion products) response directed against a peptide in a recipient patient. Such a response can be an active response, induced by administration of immunogen, or a passive response, induced by administration of antibody or primed T-cells. A cellular immune response is elicited by the presentation of polypeptide epitopes in association with Class I or Class II major histocompatibility complex (MEW) molecules to activate antigen specific CD4+T helper cells and/or CD8+ cytotoxic T cells. The response can also involve activation of monocytes, macrophages, natural killer (NK) cells, basophils, dendritic cells, astrocytes, microglia cells, eosinophils, activation or recruitment of neutrophils, or other components of innate immunity. The presence of a cell-mediated immunological response can be determined by proliferation assays (CD4+ T cells) or cytotoxic T lymphocyte (CTL) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

The terms “suppressive immune response” and “immune suppressive response” as used herein refer to a response that reduces or prevents the activation or efficiency of innate or adaptive immunity.

The term “immune tolerance” as used herein refers to any mechanism by which a potentially injurious immune response is prevented, suppressed, or shifted to a non-injurious immune response (Bach, et al., N. Eng. J. Med., 347:911-920 (2002), herein incorporated by reference in its entirety).

The term “tolerizing vaccine” as used herein is typically an antigen-specific therapy used to attenuate autoreactive T and/or B cell responses, while leaving global immune function intact.

An “immunogenic agent” or “immunogen” is capable of inducing an immunological response against itself on administration to a mammal, optionally in conjunction with an adjuvant.

The term “immune cell” as used herein refers to cells of the innate and acquired immune system including neutrophils, eosinophils, basophils, monocytes, macrophages, dendritic cells, lymphocytes including B cells, T cells, and NK cells.

As used herein, “conventional T cells” are T lymphocytes that express an αβ T cell receptor (TCR) as well as a co-receptor CD4 or CD8. Conventional T cells are present in the peripheral blood, lymph nodes, and tissues. See Roberts and Girardi, “Conventional and Unconventional T Cells”, Clinical and Basic Immunodermatology, pp. 85-104, (Gaspari and Tyring (ed.)), Springer London (2008), herein incorporated by reference in its entirety.

As used herein, “unconventional T cells” are lymphocytes that express a γδ TCR and may commonly reside in an epithelial environment, such as the skin, gastrointestinal tract, or genitourinary tract. Another subset of unconventional T cells is the invariant natural killer T (NKT) cell, which has phenotypic and functional capacities of a conventional T cell, as well as features of natural killer cells (e.g., cytolytic activity). See id.

As used herein, “Treg” refers to a regulatory T cell or cells. Regulatory T cells are a subpopulation of T cells which modulate the immune system, maintain tolerance to self-antigens, and otherwise suppress immune-stimulating or activating responses of other cells. Regulatory T cells come in many forms, with the most well-understood being those that express CD4, CD25, and Foxp3.

As used herein, “natural Treg” or “nTreg” refer to a regulatory T cell or cells that develop in the thymus.

As used herein, “induced Treg” or “iTreg” refer to a regulatory T cell or cells that develop from mature CD4+ conventional T cells outside of the thymus.

The “bioactivity” of Akt3 refers to the biological function of the Akt3 polypeptide. Bioactivity can be increased or reduced by increasing or reducing the activity of basal levels of polypeptide, increasing or reducing the avidity of basal levels of polypeptide, the quantity of the polypeptide, the ratio of Akt3 relative to one or more other isoforms of Akt (e.g., Akt1 or Akt2) of the polypeptide, increasing or reducing the expression levels of the polypeptide (including by increasing or decreasing mRNA expression of Akt3), or a combination thereof. For example, bioavailable Akt3 polypeptide is a polypeptide that has kinase activity and can bind to and phosphorylate a substrate of Akt3. Akt3 polypeptide that is not bioavailable includes Akt3 polypeptide that is mis-localized or incapable of binding to and phosphorylating Akt substrates.

As used herein, the phrase that a molecule “specifically binds” or “displays specific binding” to a target refers to a binding reaction which is determinative of the presence of the molecule in the presence of a heterogeneous population of other biologics.

Under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase enzyme-linked immunosorbent assays (ELISAs) are routinely used to select monoclonal antibodies specifically immunoreactive with a protein. See, e.g., Harlow and Lane (1988), Antibodies, A Laboratory Manual, Cold Spring Harbor Publications, New York (herein incorporated by reference in its entirety, for a description of immunoassay formats and conditions that can be used to determine specific immunoreactivity.

The terms “oligonucleotide” and “polynucleotide” generally refer to any polyribonucleotide or polydeoxribonucleotide, which may be unmodified RNA or DNA or modified RNA or DNA. Thus, for instance, “polynucleotides” as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “nucleic acid” or “nucleic acid sequence” also encompass a polynucleotide as defined above.

In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term “polynucleotide” includes DNAs or RNAs as described above that contain one or more modified bases. Thus, DNAs or RNAs with backbones modified for stability or for other reasons are “polynucleotides” as that term is intended herein. Moreover, DNAs or RNAs including unusual bases, such as inosine, or modified bases, such as tritylated bases, to name just two examples, are polynucleotides as the term is used herein.

As used herein, the term “polypeptide” refers to a chain of amino acids of any length, regardless of modification (e.g., phosphorylation or glycosylation). The term “polypeptide” includes proteins and fragments thereof. The polypeptides can be “exogenous,” meaning that they are “heterologous,” i.e., foreign to the host cell being utilized, such as human polypeptide produced by a bacterial cell. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: alanine (Ala, A), arginine (Arg, R), asparagine (Asn, N), aspartic Acid (Asp, D), cysteine (Cys, C), glutamine (Gln, Q), glutamic Acid (Glu, E), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V).

“Variant” refers to a polypeptide or polynucleotide that differs from a reference polypeptide or polynucleotide but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring, such as an allelic variant, or it may be a variant that is not known to occur naturally.

Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Trp: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more sequence identity to the polypeptide of interest.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2, or Megalign (DNASTAR). Appropriate parameters for measuring alignment, including any algorithms needed to achieve maximal alignment over the full-length of the sequences being compared, can be determined by known methods.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

The term “carrier” refers to an organic or inorganic ingredient, natural or synthetic, with which the active ingredient is combined to facilitate the application.

The term “pharmaceutically acceptable” means a non-toxic material that does not interfere with the effectiveness of the biological activity of the active ingredients.

The term “pharmaceutically-acceptable carrier” means one or more compatible solid or liquid fillers, diluents, or encapsulating substances which are suitable for administration to a human or other vertebrate animal.

The terms “effective amount” or “therapeutically effective amount” mean a dosage sufficient to provide treatment a disorder, disease, or condition being treated, or to otherwise provide a desired pharmacologic and/or physiologic effect. The precise dosage will vary according to a variety of factors, such as subject-dependent variables (e.g., age, immune system health, etc.), the disease, and the treatment being effected.

The terms “individual,” “individual,” “subject,” and “patient” are used interchangeably herein, and refer to a mammal, including, but not limited to, humans, rodents, such as mice and rats, and other laboratory animals.

As used herein, the term “motor neuron” refers to a neuron whose cell body is located in the motor cortex, brainstem, or spinal cord, and whose axon projects to the spinal cord or outside of the spinal cord to directly or indirectly control effector organs, mainly muscles and glands.

### II. Methods of Treating and Preventing Disease by Modulating Akt3 Signaling

Methods of treating and preventing disease by modulating Akt3 signaling are disclosed herein. Non-limiting examples of disease include neurodegenerative disease, cachexia, anorexia, obesity's complication, inflammatory disease, viral-induced inflammatory reaction, Gulf War Syndrome, tuberous sclerosis, retinitis pigmentosa, transplant rejection, cancer, ischemic tissue injury, traumatic tissue injury, and a combination thereof. In some embodiments, the compounds disclosed herein modulating Akt3 signaling and can be used to treat various other diseases and disorders with suspected dysfunction in PI3K/Akt signaling.

In one embodiment, the disclosed Akt3 inhibitors can be administered to a subject diagnosed with anorexia in an amount effective to promote adipogenesis and reverse extreme weight loss.

### Akt3 Modulation for the Treatment of Neurodegenerative Diseases

One embodiment provides a method of treating or preventing neurodegenerative diseases in a subject in need thereof including administering to the subject a composition comprising an Akt3 modulator in an amount effective to modulate Akt3 signaling and treat or delay the progression of the disease. Non-limiting examples of neurodegenerative diseases include Parkinson's disease, Alzheimer's disease, amyotrophic lateral sclerosis, Motor Neuron Disease, Huntington's disease, HIV-induced neurodegeneration, Lewy Body Disease, spinal muscular atrophy, prion disease, spinocerebellar ataxia, familial amyloid polyneuropathy, and a combination thereof.

Neurodegenerative diseases occur when nerve cells in the brain or peripheral nervous system lose function over time and ultimately die. In many of the neurodegenerative diseases, chronic neuroinflammation contributes to disease progression. Although current treatments may help relieve some of the physical or mental symptoms associated with neurodegenerative diseases, there are currently no ways to slow disease progression and no known cures.

While the mechanisms causing neurodegenerative processes are widely unknown, growing evidence suggests a critical role of immunity and the immune system in the pathogenesis of neurodegenerative diseases such as Alzheimer's disease, Parkinson's disease, Huntington's disease, multiple sclerosis, spinal muscular atrophy, and amyotrophic lateral sclerosis (ALS). Regulatory T cells (Tregs) are a subset of CD4+ T cells that suppress immune responses and are essential mediators of self-tolerance and immune homeostasis (Sakaguchi, et al., Cell, 133, 775-787 (2008); incorporated herein by reference in its entirety). Several lines of evidence suggest that Tregs play an important role in the progression of neurodegenerative diseases. It has been discovered that Akt3 modulates the suppressive function of natural Tregs and the polarization of induced Tregs and, therefore, modulating Akt3 in immune cells can modulate immune responses. More specifically, activating Akt3 in immune cells leads to increased immune suppressive responses, while inhibiting Akt3 in immune cells leads to decreased immune suppressive responses. Without being bound by any one theory, it is believed that modulating Akt3 signaling in immune cells can be used for the treatment and prevention of neurodegenerative diseases.

One embodiment provides a method of treating or preventing neurodegenerative disease in a subject in need thereof by administering to the subject an Akt3 activator in an amount effective to induce an immune suppressive response and treat or delay the progression of the disease. In one embodiment, the Akt3 activator modulates an immune response by increasing a suppressive function of immune suppressive cells. In one embodiment, Akt3 is selectively activated in immune cells. Exemplary immune cells include, but are not limited to, T cells, B cells, macrophages, and glial cells, such as astrocytes, microglia, and oligodendrocytes. In a preferred embodiment, Akt3 is activated in Tregs. The Akt3 activators can also be used to increase or promote the activity or production of Tregs, increase the production of cytokines, such as IL-10, from Tregs, increase the differentiation of Tregs, increase the number of Tregs, or increase the survival of Tregs.

Another embodiment provides a method of treating or preventing neurodegenerative disease in a subject in need thereof by administering to the subject an Akt3 inhibitor in an amount effective to inhibit an immune suppressive response and treat or prevent the progression of the disease. In one embodiment, the Akt3 inhibitor modulates an immune response by decreasing an immune suppressive response or increasing an immune stimulatory response. In one embodiment, Akt3 is selectively inhibited in immune cells. Exemplary immune cells include but are not limited to T cells, B cells, macrophages, and glial cells, such as astrocytes, microglia, and oligodendrocytes. In a preferred embodiment, Akt3 is inhibited in Tregs.

1. Subjects to be Treated

a. Amyotrophic Lateral Sclerosis (ALS)

In one embodiment, the disclosed Akt3 modulators can treat or prevent ALS. ALS, also called Lou Gehrig's disease, is a progressive neurodegenerative disease that affects motor neurons in the brain and spinal cord. Symptoms of ALS include, but are not limited to, difficulty speaking, swallowing, walking, moving, and breathing. ALS usually affects men and women between the ages of 40 and 70. There are two different types of ALS, sporadic and familial. Sporadic, which is the most common form of the disease in the U.S., accounts for 90 to 95 percent of all cases. Familial ALS has been associated with mutations in Cu/Zn superoxide dismutase (SOD1). Oxidative stress, mitochondrial dysfunction, excitotoxicity, protein aggregation, endoplasmic reticulum stress, impairment of axonal transport, dysregulation of neuronal-glial interactions, and apoptosis have all been demonstrated to contribute to motor neuron injury in the presence of mutant SOD1.

Without being bound by any one theory, it is believed that Treg dysfunction plays a role in the development of ALS and administration of an Akt3 modulator can treat or prevent the progression of ALS. It has been discovered that some subjects with rapidly progressing ALS have a deficiency of the Treg master transcription factor FOXP3 which leads to impairment of Treg suppressive function. One embodiment provides a method of treating ALS in a subject in need thereof by administering an Akt3 activator to a subject in need thereof in an amount effective to activate Akt3 in immune cells and induce immune suppressive responses. In a preferred embodiment, Akt3 is activated in Tregs.

In one embodiment, administration of Akt3 activators to a subject having ALS slows disease progression and prolongs the subject's survival.

Other motor neuron diseases that can be treated or prevented using the disclosed Akt3 activators include, but are not limited to, progressive bulbar palsy, pseudobulbar palsy, primary lateral sclerosis, spinal muscular atrophy, and post-polio syndrome.

b. Parkinson's Disease

Parkinson's disease is a neurodegenerative disorder that predominantly affects dopamine-producing neurons in a specific area of the brain called substantia nigra. Parkinson's disease is a progressive disease that worsens over time as more neurons become impaired or die. The cause of neuronal death in Parkinson's is not known. Symptoms of Parkinson's disease include but are not limited to tremors in hands, arms, legs, jaw, or head, stiffness of the limbs and trunk, slowness of movement, and impaired balance and coordination.

One embodiment provides a method of treating Parkinson's disease by administering an Akt3 modulator to a subject in need thereof in an amount effective to activate or inhibit Akt3 in immune cells and induce an immune suppressive response. In one embodiment, administration of Akt3 activators to a subject having Parkinson's disease will slow or stop disease progression to unaffected areas of the brain.

In one embodiment, the disclosed Akt3 activators can be administered to a subject prophylactically if the subject has a family history of Parkinson's disease or other neurodegenerative diseases. The Akt3 activators can protect neurons from disease induction or slow down the induction of the disease.

c. Huntington's Disease

Huntington's disease is a progressive neurodegenerative disease. The disease is characterized by the progressive breakdown of nerve cells in the brain. Symptoms of Huntington's disease include, but are not limited to, involuntary movement problems and impairments in voluntary movement such as involuntary jerking, muscle rigidity, slow or abnormal eye movements, impaired gait, posture, and balance, difficulty with the physical production of speech or swallowing; cognitive impairments such as difficulty organizing, prioritizing, or focusing on tasks, lack of flexibility or the tendency to get stuck on a thought, behavior, or action, lack of impulse control, lack of awareness of one's own behaviors and abilities, slowness in processing thoughts or finding words, and difficulty in learning new information; and psychiatric disorders such as depression. In one embodiment, the disclosed Akt3 modulators can lessen or slow down the progression of symptoms of Huntington's disease.

One embodiment provides a method of treating Huntington's disease in a subject in need thereof by administering an Akt3 modulator to the subject in an amount effective to activate or inhibit Akt3 in immune cells and induce an immune suppressive response. In one embodiment, Akt3 modulators can slow down or stop the progression of disease symptoms in subjects with Huntington's disease. In another embodiment, Akt3 modulators can alter the Treg/Th17 balance.

Huntington's disease is largely genetic; every child of a parent with Huntington's disease has a 50/50 chance of inheriting the disease. In one embodiment, subjects with a familial history of Huntington's disease can be prophylactically administered one of the disclosed Akt3 modulators before symptoms of the disease appear to prevent or slow down the manifestation of disease symptoms.

d. Alzheimer's Disease

Alzheimer's disease is a progressive disorder that causes brain cells to degenerate and eventually die. Alzheimer's disease is the most common cause of dementia—a continuous decline in thinking, behavioral, and social skills that disrupts a person's ability to function independently. Symptoms of Alzheimer's disease include, but are not limited to, memory loss, impairment in thinking and reasoning abilities, difficulty in making judgments and decisions, and changes in personality and behavior. While the exact cause of Alzheimer's disease is not fully understood, it is believed that the core problem is dysfunctionality in brain proteins which disrupt neuronal function and unleash a series of toxic events. The damage most often starts in the region of the brain that controls memory, but the process begins years before the first symptoms. The loss of neurons spreads in a somewhat predictable pattern to other regions of the brains. By the late stage of the disease, the brain has shrunk significantly. Beta-amyloid plaques and tau protein tangles are most often attributed with the bulk of the damage and dysfunctionality of neurons in Alzheimer's disease.

One embodiment provides a method of treating Alzheimer's disease in a subject by administering an Akt3 activator to the subject in an amount effective to activate Akt3 in Tregs and activate downstream neuroprotective pathways in the brain. In another embodiment, subjects are administered an effective amount of an Akt3 activator to reduce or eliminate symptoms of Alzheimer's disease or to slow down disease progression.

Another embodiment provides a method of treating or preventing the progression of Alzheimer's disease in a subject by administering an Akt3 inhibitor to the subject in an amount effective to inhibit Akt3 in Tregs and induce an immune response or decrease an immune suppressive response. In one embodiment, inhibition of Akt3 in Tregs leads to beta-amyloid plaque clearance, mitigation of neuroinflammatory response, and reversal of cognitive decline.

In one embodiment, subjects with a family history of Alzheimer's disease can be prophylactically administered an Akt3 modulator to prevent or slow down the manifestation of Alzheimer's disease.

e. Spinal Muscular Atrophy

Spinal muscular atrophy (SMA) is a group of chronic neuromuscular disorders that are characterized by progressive loss of motor neurons and muscle wasting. SMA is commonly classified in four types that vary in severity and the life stage during which the disease manifests. These types are:
SMA1 or Werdnig-Hoffmann disease, which manifests during age 0-6 months (“infantile” SMA);
SMA2 or Dubowitz disease, which manifests during age 6-18 months (“intermediate” SMA);
SMA3 or Kugelberg-Welander disease, which manifests after age 1 year (“juvenile” SMA); and
SMA4, which manifests during adulthood (“adult-onset” SMA).

The most severe form of SMA1 is sometimes termed SMA0 (“severe infantile” SMA). Signs and symptoms of SMA vary according to type, but the most common include, but are not limited to, limpness or tendency to flop, difficulty sitting, standing, or walking, loss of strength in respiratory muscles, twitching, and difficulty eating and swallowing. All types of SMA have been linked to exonal deletion and/or point mutations in the SMN1 gene, preventing expression of the SMN protein. Depending on the type, SMA can be treated with various gene therapies, assisted nutrition and respiration, orthopedics, and combinations thereof. Neuroprotective drugs are promising as a way to stabilize motor neuron loss, but currently available candidates have yet to successfully advance through clinical trials. Therefore, more candidate neuroprotective drugs are needed for treatment of SMA,

One embodiment provides a method of treating SMA in a subject by administering an Akt3 activator to the subject in an amount effective to enable survival of motor neurons. In another embodiment, subjects are administered an effective amount of an Akt3 activator to reduce or eliminate symptoms of SMA or to slow down disease progression.

### Akt3 Inhibition for the Treatment of Extreme Weight Loss

Methods of treating or preventing extreme weight loss associated with diseases and disorders such as cachexia and anorexia are disclosed herein. An exemplary method includes inhibiting Akt3 in subjects in need thereof. Without being bound by any one theory, it is believed that Akt3 plays an important role in adipogenesis. White adipogenesis requires activation of a transcriptional cascade involving the sequential induction of a number of transcription factors including, but not limited to, FOXO1, several members of the C/EBP family, and PPARγ. FOXO1 is an essential negative regulator of adipogenesis and is primarily controlled through phosphorylation/acetylation on multiple residues by enzymes including Akt. FOXO1 can also be controlled by the serine/threonine protein kinase SGK1. SGK1 is downstream of PI3K and can inhibit FOXO1 upon phosphorylation. SGK1 is regulated by the serine/threonine protein kinase WNK1, which can also be regulated by Akt and SGK1. Akt3 suppresses adipogenesis through phosphorylation of WNK1, leading to downregulation of SGK1 activity and SGK-1-mediated inhibition of FOXO1. In one embodiment, inhibition of Akt3 in Tregs can promote adipogenesis and reverse disease-induced weight loss.

1. Subjects to be Treated

a. Cachexia

Cachexia, or wasting syndrome, is a multifactorial syndrome characterized by an ongoing loss of skeletal muscle that cannot be fully reversed by conventional nutritional support and leads to progressive functional impairment. Cachexia is so destructive that it taps into other sources of energy, namely skeletal muscle and adipose tissue, when the body senses lack of nutrition. It is associated with a reduction in ability to fight infection, treatment tolerance, response to therapy, quality of life, and duration of survival.

In one embodiment, the cachexia is caused by a chronic disease such as, but not limited to, AIDS, celiac disease, chronic obstructive pulmonary disease, multiple sclerosis, rheumatoid arthritis, congestive heart failure, tuberculosis, familial amyloid polyneuropathy, Crohn's disease, untreated and severe type 1 diabetes, anorexia nervosa, hyperthyroidism, and hormonal deficiency.

One embodiment provides a method of treating cachexia in a subject in need thereof by administering an Akt3 inhibitor to the subject in an amount effective to reduce symptoms of cachexia. Another embodiment provides a method of promoting weight gain in a subject in need thereof by administering an Akt3 inhibitor to the subject in an amount effective to promote adipogenesis in the subject. In some embodiments, the compound disclosed herein is used for treating cachexia by modulating Akt3 and not by modulating T regulatory cells.

In one embodiment, a subject suspected of being susceptible for cachexia (for example, subjects who have been diagnosed with other diseases) can be prophylactically administered an Akt3 inhibitor to prevent or slow down the manifestation of cachexia syndrome.

b. Anorexia

Anorexia nervosa is an eating disorder characterized by weight loss or the lack of weight gain in growing children, difficulties maintaining an appropriate body weight for height, age, and stature, and, often, distorted body image. One of the first goals of treatment for anorexia is the restoration of a normal body weight. In some embodiments, the compound of Formula I disclosed herein inhibits Akt3, which has been overactivated by estradiol, the levels of which are increased in subjects with anorexia. In some embodiments, the compound of Formula I disclosed herein can be used to treat anorexia.

### Akt3 Modulation for Treating Obesity and Obesity's Complications

In some embodiments, the compound disclosed herein modulating Akt3 is used for treating Obesity and/or Obesity's complications. In some embodiments, the obesity's complication is selected from the group consisting of glucose intolerance, hepatic steatosis, dyslipidemia, and a combination thereof. In some embodiments, the compound disclosed herein is used for treating Obesity and/or Obesity's complications by modulating Akt3 and not by modulating T regulatory cells.

### Akt3 Modulation for Treatment of Inflammatory Diseases

Akt3 signaling has been linked to the chronic or acute inflammation that contributes to inflammatory diseases. One embodiment provides a method of treating or preventing an inflammatory disease in a subject in need thereof including administering to the subject a composition comprising an Akt3 modulator in an amount effective to modulate Akt3 signaling and treat or delay the progression of the disease. In some embodiments, the Akt3 modulator activates Akt3 signaling and/or increases Treg activity or production, resulting in an immunosuppressive effect.

Non-limiting examples of inflammatory disease include atopic dermatitis, allergy, asthma, and a combination thereof.

### Akt3 Modulation for Treatment Viral-Induced Inflammatory Reaction

Akt3 signaling has been linked to the acute immune responses that contribute to viral-induced inflammatory diseases, such as severe acute respiratory syndrome (“SARS”) and coronavirus disease 2019 (“COVID-19”). Therefore, in one embodiment, a method of treating a viral-induced inflammatory disease in a subject in need thereof includes administering to the subject an Akt3 modulator in an amount effective to reverse or slow down the progression of the disease.

### Akt3 Modulation for the Treatment of Cancer

In some embodiments, a method of treating or preventing cancer in a subject in need thereof is provided, including modulating Akt3 signaling through administering to the subject an effective amount of a compound disclosed herein. In some embodiments, the compound disclosed herein inhibits Akt3 signaling and/or decreases Treg activity or production, resulting in an immune response-activating effect.

In some embodiments, the cancer is selected from the group consisting of adult T-cell leukemia/lymphoma, bladder cancer, brain cancer, breast cancer, cervical cancer, colorectal cancer, esophageal cancer, kidney cancer, liver cancer, lung cancer, nasopharyngeal cancer, pancreatic cancer, prostate cancer, skin cancer, stomach cancer, uterine cancer, ovarian cancer, testicular cancer, and a combination thereof.

In some embodiments, the compounds and compositions disclosed herein are useful for treating leukemia. In some embodiments, the compounds and compositions disclosed herein that inhibit Akt3 are useful for treating leukemia. In these embodiments, the compounds and compositions disclosed herein that inhibit Akt3 are useful in vivo and ex vivo as immune response-stimulating therapeutics. The ability to inhibit Akt3 and thereby inhibit or reduce Treg-mediated immune suppression enables a more robust immune response. In some embodiments, the compounds and compositions disclosed herein are also useful to stimulate or enhance immune-stimulating or -activating responses involving T cells. In some embodiments, the compounds and compositions disclosed herein are useful for stimulating or enhancing an immune response in a host for treating leukemia by selectively inhibiting Akt3. In these embodiments, the compounds and compositions disclosed herein can be administered to a subject in an amount effective to stimulate T cells in the subject. The types of leukemia that can be treated with the compounds and compositions as disclosed herein include, but are not limited to, acute myeloid leukemia (AML), chronic myeloid leukemia (CML), acute lymphocytic leukemia (ALL), chronic lymphocytic leukemia (CLL), adult T-cell leukemia/lymphoma (ATLL) and chronic myelomonocytic leukemia (CMML).

In some embodiments, ATLL is almost exclusively diagnosed in adults, with a median age in the mid-60s. In some embodiments, there are four types of ATLL: (1) acute, (2) chronic, (3) smouldering, and (4) lymphomatous. In some embodiments, acute ATLL is the most common form, and is characterized by high white blood cell count, hypercalcemia, organomegaly, and high lactose dehydrogenase. In some embodiments, lymphomatous ATLL manifests in the lymph nodes with less than 1% circulating lymphocytes. In some embodiments, chronic and smouldering ATLL are characterized by a less aggressive clinical course and allow for long-term survival. In some embodiments, the four-year survival rate for acute and lymphomatous ATLL is less than 5%. In some embodiments, chronic and smouldering forms of ATLL have four-year survival rates of 26.9% and 62%, respectively. In some embodiments, the adult T-cell leukemia/lymphoma is caused by human T-cell lymphotropic virus (HTLV-1).

In some embodiments, the compounds and compositions disclosed herein are useful for treating ATLL. In some embodiments, the compounds and compositions disclosed herein that inhibit Akt3 are useful for treating ATLL. In some embodiments, Tregs expressing CD25 and FoxP3 may transform into ATLL cells. In some embodiments, ATLL cells display an activated helper/inducer T-cell phenotype but exhibit strong immunosuppressive activity. In some embodiments, the compounds and compositions disclosed herein that inhibit Akt3 reduce the immunosuppressive response of the ATLL cells. In other embodiments, the compounds and compositions disclosed herein that inhibit Akt3 increase an immune stimulatory response to overcome the strong immunosuppressive activity of ATLL cells.

In some embodiments, the compounds and compositions disclosed herein that are useful for treating leukemia or ATLL reduce or inhibit an immune suppressive response, such as, but not limited to an immune suppressive function of natural Treg (nTreg) cells and induction of conventional T cells into induced Treg (iTreg). In these embodiments, the immune suppressive function of nTreg cells that is reduced or inhibited is the secretion of one or more anti-inflammatory cytokines, such as, but not limited to IL10, TGFβ, or a combination thereof. In some embodiments, methods for treating leukemia or adult T-cell leukemia/lymphoma include administering to a subject a second active agent, such as, but not limited to, an anti-nausea drug, a chemotherapeutic drug, or a potentiating agent (e.g., cyclophosphamide).

### Other Indications

In some embodiments, a compound disclosed herein modulates Akt3 and is used for treating Gulf War Syndrome, tuberous sclerosis, retinitis pigmentosa, or transplant rejection. In some embodiments, the transplant rejection is Graft-versus-Host disease. In some embodiments, the compound disclosed herein is used for treating retinitis pigmentosa by modulating Akt3 and not by modulating T regulatory cells. In some embodiments, the compound disclosed herein is used for treating ischemic tissue injury or traumatic tissue injury. In some embodiments, the ischemic tissue injury or traumatic tissue injury is the ischemic tissue injury or traumatic tissue injury of the brain.

### III. Methods of Modulating Akt3

Akt3, also referred to as RAC-gamma serine/threonine-protein kinase, is an enzyme that in humans is encoded by the Akt3 gene. Akt kinases are known to be regulators of cell signaling in response to insulin and growth factors and are associated with a broad range of biological processes including cell proliferation, differentiation, apoptosis, and tumorigenesis, as well as glycogen synthesis and glucose uptake. Akt3 has been shown to be stimulated by platelet-derived growth factor (PDGF), insulin, and insulin-like growth factor 1 (IGF 1).

Akt3 kinase activity mediates serine and/or threonine phosphorylation of a range of downstream substrates. Nucleic acid sequences for Akt3 are known in the art. See, for example, Genbank accession no. AF124141.1: Homo sapiens protein kinase B gamma mRNA, complete cds, which is specifically incorporated by reference in its entirety, and provides the following nucleic acid sequence:

(SEQ ID NO: 1)
AGGGGAGTCATCATGAGCGATGTTACCATTGTGAAGGAAGGTTGGGTTCA
GAAGAGGGGAGAATATATAAAAAACTGGAGGCCAAGATACTTCCTTTTGA
AGACAGATGGCTCATTCATAGGATATAAAGAGAAACCTCAAGATGTGGAT
TTACCTTATCCCCTCAACAACTTTTCAGTGGCAAAATGCCAGTTAATGAA
AACAGAACGAGCAAAGCCAAACACATTTATAATCAGATGTCTCCAGTGGA
CTACTGTTATAGAGAGAACATTTCATGTAGATACTCCAGAGGAAAGGGAA
GAATGGACAGAAGCTATCCAGGCTGTAGCAGACAGACTGCAGAGGCAAGA
AGAGGAGAGAATGAATTGTAGTCCAACTTCACAAATTGATAATATAGGAG
AGGAAGAGATGGATGCCTCTACAACCCATCATAAAAGAAAGACAATGAAT
GATTTTGACTATTTGAAACTACTAGGTAAAGGCACTTTTGGGAAAGTTAT
TTTGGTTCGAGAGAAGGCAAGTGGAAAATACTATGCTATGAAGATTCTGA
AGAAAGAAGTCATTATTGCAAAGGATGAAGTGGCACACACTCTAACTGAA
AGCAGAGTATTAAAGAACACTAGACATCCCTTTTTAACATCCTTGAAATA
TTCCTTCCAGACAAAAGACCGTTTGTGTTTTGTGATGGAATATGTTAATG
GGGGCGAGCTGTTTTTCCATTTGTCGAGAGAGCGGGTGTTCTCTGAGGAC
CGCACACGTTTCTATGGTGCAGAAATTGTCTCTGCCTTGGACTATCTACA
TTCCGGAAAGATTGTGTACCGTGATCTCAAGTTGGAGAATCTAATGCTGG
ACAAAGATGGCCACATAAAAATTACAGATTTTGGACTTTGCAAAGAAGGG
ATCACAGATGCAGCCACCATGAAGACATTCTGTGGCACTCCAGAATATCT
GGCACCAGAGGTGTTAGAAGATAATGACTATGGCCGAGCAGTAGACTGGT
GGGGCCTAGGGGTTGTCATGTATGAAATGATGTGTGGGAGGTTACCTTTC
TACAACCAGGACCATGAGAAACTTTTTGAATTAATATTAATGGAAGACAT
TAAATTTCCTCGAACACTCTCTTCAGATGCAAAATCATTGCTTTCAGGGC
TCTTGATAAAGGATCCAAATAAACGCCTTGGTGGAGGACCAGATGATGCA
AAAGAAATTATGAGACACAGTTTCTTCTCTGGAGTAAACTGGCAAGATGT
ATATGATAAAAAGCTTGTACCTCCTTTTAAACCTCAAGTAACATCTGAGA
CAGATACTAGATATTTTGATGAAGAATTTACAGCTCAGACTATTACAATA
ACACCACCTGAAAAATATGATGAGGATGGTATGGACTGCATGGACAATGA
GAGGCGGCCGCATTTCCCTCAATTTTCCTACTCTGCAAGTGGACGAGAAT
AAGTCTCTTTCATTCTGCTACTTCACTGTCATCTTCAATTTATTACTGAA
AATGATTCCTGGACATCACCAGTCCTAGCTCTTACACATAGCAGGGGCAC
CTTCCGACATCCCAGACCAGCCAAGGGTCCTCACCCCTCGCCACCTTTCA
CCCTCATGAAAACACACATACACGCAAATACACTCCAGTTTTTGTTTTTG
CATGAAATTGTATCTCAGTCTAAGGTCTCATGCTGTTGCTGCTACTGTCT
TACTATTA.

Amino acid sequences are also known in the art. See, for example, UniProtKB/Swiss-Prot accession no. Q9Y243 (Akt3_HUMAN), which is specifically incorporated by reference in its entirety and provides the following amino acid sequence:

(SEQ ID NO: 2)
MSDVTIVKEGWVQKRGEYIKNWRPRYFLLKTDGSFIGYKEKPQDVDLPYP
LNNFSVAKCQLMKTERPKPNTFIIRCLQWTTVIERTFHVDTPEEREEWTE
AIQAVADRLQRQEEERMNCSPTSQIDNIGEEEMDASTTHHKRKTMNDFDY
LKLLGKGTFGKVILVREKASGKYYAMKILKKEVIIAKDEVAHTLTESRVL
KNTRHPFLTSLKYSFQTKDRLCFVMEYVNGGELFFHLSRERVFSEDRTRF
YGAEIVSALDYLHSGKIVYRDLKLENLMLDKDGHIKITDFGLCKEGITDA
ATMKTFCGTPEYLAPEVLEDNDYGRAVDWWGLGVVMYEMMCGRLPFYNQD
HEKLFELILMEDIKFPRTLSSDAKSLLSGLLIKDPNKRLGGGPDDAKEIM
RHSFFSGVNWQDVYDKKLVPPFKPQVTSETDTRYFDEEFTAQTITITPPE
KYDEDGMDCMDNERRPHFPQFSYSASGRE.

The domain structure of Akt3 is reviewed in Romano, Scientifica, Volume 2013 (2013), Article ID 317186, 12 pages (incorporated herein by reference in its entirety), and includes an N-terminal pleckstrin homology domain (PH), followed by a catalytic kinase domain (KD), and the C-terminal regulatory hydrophobic region. The catalytic and regulatory domains are both important for the biological actions mediated by Akt protein kinases and exhibit the maximum degree of homology among the three Akt isoforms. The PH domain binds lipid substrates, such as phosphatidylinositol (3,4) diphosphate (PIP2) and phosphatidylinositol (3,4,5) triphosphate (PIP3). The ATP binding site is situated approximately in the middle of the catalytic kinase domain, which has a substantial degree of homology with the other components of the AGC kinases family, such as p70 S6 kinase (S6K) and p90 ribosomal S6 kinase (RSK), protein kinase A (PKA), and protein kinase B (PKB). The hydrophobic regulatory moiety is a typical feature of the AGC kinases family. With reference to SEQ ID NO:2, Akt 3 is generally considered to have the molecule processing and domain structure outlined as follows.

Molecule Processing:

| Feature key | Position(s) | Length | Description |
|---|---|---|---|
| Initiator methionine | 1 | 1 | Removed |
| Chain | 2-479 | 478 | Akt3 |

Regions:

| Feature key | Position(s) | Length | Description |
|---|---|---|---|
| Domain | 5-107 | 103 | PH |
| Domain | 148-405 | 258 | Protein kinase |
| Domain | 406-479 | 74 | AGC-kinase C-terminal |
| Nucleotide binding | 154-162 | 9 | ATP |

Sites:

| Feature key | Position(s) | Length | Description |
|---|---|---|---|
| Active site | 271 | 1 | Proton acceptor |
| Binding site | 177 | 1 | ATP |

The initiator methionine of SEQ ID NO:2 is disposable for Akt3 function. Therefore, in some embodiments, the compound directly or indirectly modulates expression or bioavailability of an Akt3 having the following amino acid sequence:

(SEQ ID NO: 3)
SDVTIVKEGWVQKRGEYIKNWRPRYFLLKTDGSFIGYKEKPQDVDLPYPL
NNFSVAKCQLMKTERPKPNTFIIRCLQWTTVIERTFHVDTPEEREEWTEA
IQAVADRLQRQEEERMNCSPTSQIDNIGEEEMDASTTHHKRKTMNDFDYL
KLLGKGTFGKVILVREKASGKYYAMKILKKEVIIAKDEVAHTLTESRVLK
NTRHPFLTSLKYSFQTKDRLCFVMEYVNGGELFFHLSRERVFSEDRTRFY
GAEIVSALDYLHSGKIVYRDLKLENLMLDKDGHIKITDFGLCKEGITDAA
TMKTFCGTPEYLAPEVLEDNDYGRAVDWWGLGVVMYEMMCGRLPFYNQDH
EKLFELILMEDIKFPRTLSSDAKSLLSGLLIKDPNKRLGGGPDDAKEIMR
HSFFSGVNWQDVYDKKLVPPFKPQVTSETDTRYFDEEFTAQTITITPPEK
YDEDGMDCMDNERRPHFPQFSYSASGRE.

Two specific sites, one in the kinase domain (Thr-305 with reference to SEQ ID NO:2) and the other in the C-terminal regulatory region (Ser-472 with reference to SEQ ID NO:2), need to be phosphorylated for full activation of Akt3. Interaction between the PH domain of Akt3 and TCL1A enhances Akt3 phosphorylation and activation. IGF-1 leads to the activation of Akt3, which may play a role in regulating cell survival.

Compositions and methods of their use for selectively modulating Akt3 activity are disclosed herein. Methods of using the disclosed Akt3 modulators to treat or prevent various diseases are also described.

A. Akt3 Activator Compounds

Compositions for selectively activating Akt3 are provided herein. Exemplary Akt3 activators are described in International Application No. PCT/US2018/49715 (incorporated herein by reference in its entirety) and are described below.

One embodiment provides a compound of Formula I:

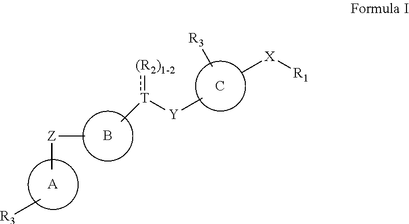

or a pharmaceutically acceptable enantiomer, salt, or solvate thereof, wherein:

rings A, B, and C are independently six-membered aryl or N-containing heteroaryl mono- or bicyclic ring systems containing zero or more N-atoms, such as phenyl, pyridine, pyrimidine, pyridazine, pyrazine, triazine, quinoline, quinazoline, isoquinoline, naphthalene, naphthyridine, indole, isoindole, cinnoline, phthalazine, quinoxaline, pteridine, purine, and benzimidazole;

R₁ is selected from —(C₁-C₃₀)-alkyl, —(C₃-C₁₂)-cycloalkyl, —(C₃-C₁₂)-heterocycloalkyl, —(C₆-C₂₀)-aryl, or —(C₃-C₂₀)-heteroaryl groups optionally substituted by one or more substituents selected from —(C₁-C₁₂)-alkyl, —(C₃-C₁₂)-cycloalkyl, —(C₃-C₁₂)-heterocycloalkyl, —O—(C₁-C₁₂)-alkyl, —O—(C₁-C₁₂)-alkyl-(C₆-C₂₀)-aryl, —O—(C₃-C₁₂)-cycloalkyl, —S—(C₁-C₁₂)-alkyl, —S—(C₃-C₁₂)-cycloalkyl, —COO—(C₁-C₁₂)-alkyl, —COO—(C₃-C₁₂)-cycloalkyl, —CONH—(C₁-C₁₂)-alkyl, —CONH—(C₃-C₁₂)-cycloalkyl, —CO—(C₁-C₁₂)-alkyl, —CO—(C₃-C₁₂)-cycloalkyl, —N—[(C₁-C₁₂)-alkyl]₂, —(C₆-C₂₀)-aryl, —(C₆-C₂₀)-aryl-(C₁-C₁₂)-alkyl, —(C₆-C₂₀)-aryl-O—(C₁-C₁₂)-alkyl, —(C₃-C₂₀)-heteroaryl, —(C₃-C₂₀)-heteroaryl-(C₁-C₁₂)-alkyl, —(C₃-C₂₀)-heteroaryl-O—(C₁-C₁₂)-alkyl, —COOH, —OH, —SH, —SO₃H, —CN, —NH₂, or a halogen;

X, Y, and Z are independently selected from ═O, —NH, —S, —N—(C₁-C₃₀)-alkyl, or —(C₁-C₃₀)-aryl;

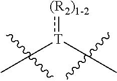

is —CH((C₁-C₃₀)-alkyl))—, —(C═O)—, —CH(OH), —SO₂—, —SO—, or —CH(SOCH₃)—; and

R₃ is selected from —(C₁-C₃₀)-alkyl, —(C₃-C₁₂)-cycloalkyl, —(C₃-C₁₂)-heterocycloalkyl, —(C₆-C₂₀)-aryl, or —(C₃-C₂₀)-heteroaryl groups optionally substituted by one or more substituents selected from —(C₁-C₁₂)-alkyl, —(C₃-C₁₂)-cycloalkyl, —(C₃-C₁₂)-heterocycloalkyl, —O—(C₁-C₁₂)-alkyl, —O—(C₁-C₁₂)-alkyl-(C₆-C₂₀)-aryl, —O—(C₃-C₁₂)-cycloalkyl, —S—(C₁-C₁₂)-alkyl, —S—(C₃-C₁₂)-cycloalkyl, —COO—(C₁-C₁₂)-alkyl, —COO—(C₃-C₁₂)-cycloalkyl, —CONH—(C₁-C₁₂)-alkyl, —CONH—(C₃-C₁₂)-cycloalkyl, —CO—(C₁-C₁₂)-alkyl, —CO—(C₃-C₁₂)-cycloalkyl, —N—[(C₁-C₁₂)-alkyl]₂, —(C₆-C₂₀)-aryl, —(C₆-C₂₀)-aryl-(C₁-C₁₂)-alkyl, —(C₆-C₂₀)-aryl-O—(C₁-C₁₂)-alkyl, —(C₃-C₂₀)-heteroaryl, —(C₃-C₂₀)-heteroaryl-(C₁-C₁₂)-alkyl, —(C₃-C₂₀)-heteroaryl-O—(C₁-C₁₂)-alkyl, —COOH, —OH, —SH, —SO₃H, —CN, —NH₂, or a halogen.

Another embodiment provides a compound of Formula II:

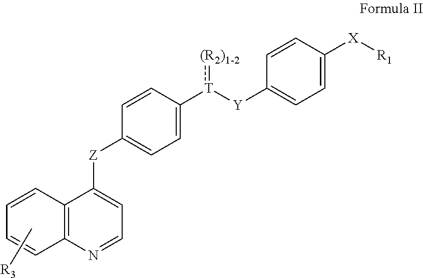

or a pharmaceutically acceptable enantiomer, salt, or solvate thereof, wherein:

R₁ is selected from —(C₁-C₃₀)-alkyl, —(C₃-C₁₂)-cycloalkyl, —(C₃-C₁₂)-heterocycloalkyl, —(C₆-C₂₀)-aryl, or —(C₃-C₂₀)-heteroaryl groups optionally substituted by one or more substituents selected from —(C₁-C₁₂)-alkyl, —(C₃-C₁₂)-cycloalkyl, —(C₃-C₁₂)-heterocycloalkyl, —O—(C₁-C₁₂)-alkyl, —O—(C₁-C₁₂)-alkyl-(C₆-C₂₀)-aryl, —O—(C₃-C₁₂)-cycloalkyl, —S—(C₁-C₁₂)-alkyl, —S—(C₃-C₁₂)-cycloalkyl, —COO—(C₁-C₁₂)-alkyl, —COO—(C₃-C₁₂)-cycloalkyl, —CONH—(C₁-C₁₂)-alkyl, —CONH—(C₃-C₁₂)-cycloalkyl, —CO—(C₁-C₁₂)-alkyl, —CO—(C₃-C₁₂)-cycloalkyl, —N—[(C₁-C₁₂)-alkyl]₂, —(C₆-C₂₀)-aryl, —(C₆-C₂₀)-aryl-(C₁-C₁₂)-alkyl, —(C₆-C₂₀)-aryl-O—(C₁-C₁₂)-alkyl, —(C₃-C₂₀)-heteroaryl, —(C₃-C₂₀)-heteroaryl-(C₁-C₁₂)-alkyl, —(C₃-C₂₀)-heteroaryl-O—(C₁-C₁₂)-alkyl, —COOH, —OH, —SH, —SO₃H, —CN, —NH₂, or a halogen;

X, Y, and Z are independently selected from —O, —NH, —S, —N—(C₁-C₃₀)-alkyl, or —(C₁-C₃₀)-aryl;

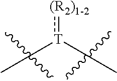

is —CH((C₁-C₃₀)-alkyl)), —(C═O)—, —CH(OH), —SO₂—, —SO—, or —CH(SOCH₃)—; and

R₃ is selected from —(C₁-C₃₀)-alkyl, —(C₃-C₁₂)-cycloalkyl, —(C₃-C₁₂)-heterocycloalkyl, —(C₆-C₂₀)-aryl, or —(C₃-C₂₀)-heteroaryl groups optionally substituted by one or more substituents selected from —(C₁-C₁₂)-alkyl, —(C₃-C₁₂)-cycloalkyl, —(C₃-C₁₂)-heterocycloalkyl, —O—(C₁-C₁₂)-alkyl, —O—(C₁-C₁₂)-alkyl-(C₆-C₂₀)-aryl, —O—(C₃-C₁₂)-cycloalkyl, —S—(C₁-C₁₂)-alkyl, —S—(C₃-C₁₂)-cycloalkyl, —COO—(C₁-C₁₂)-alkyl, —COO—(C₃-C₁₂)-cycloalkyl, —CONH—(C₁-C₁₂)-alkyl, —CONH—(C₃-C₁₂)-cycloalkyl, —CO—(C₁-C₁₂)-alkyl, —CO—(C₃-C₁₂)-cycloalkyl, —N—[(C₁-C₁₂)-alkyl]₂, —(C₆-C₂₀)-aryl, —(C₆-C₂₀)-aryl-(C₁-C₁₂)-alkyl, —(C₆-C₂₀)-aryl-O—(C₁-C₁₂)-alkyl, —(C₃-C₂₀)-heteroaryl, —(C₃-C₂₀)-heteroaryl-(C₁-C₁₂)-alkyl, —(C₃-C₂₀)-heteroaryl-O—(C₁-C₁₂)-alkyl, —COOH, —OH, —SH, —SO₃H, —CN, —NH₂, or a halogen.

Another embodiment provides a compound of Formula III:

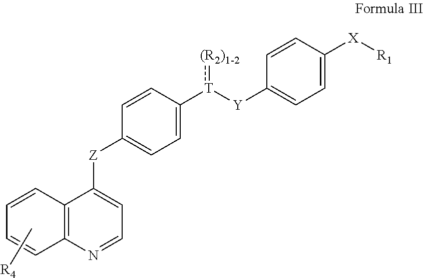

or a pharmaceutically acceptable enantiomer, salt, or solvate thereof, wherein:

R₁ is selected from —(C₁-C₃₀)-alkyl, —(C₃-C₁₂)-cycloalkyl, —(C₃-C₁₂)-heterocycloalkyl, —(C₆-C₂₀)-aryl, or —(C₃-C₂₀)-heteroaryl groups optionally substituted by one or more substituents selected from —(C₁-C₁₂)-alkyl, —(C₃-C₁₂)-cycloalkyl, —(C₃-C₁₂)-heterocycloalkyl, —O—(C₁-C₁₂)-alkyl, —O—(C₁-C₁₂)-alkyl-(C₆-C₂₀)-aryl, —O—(C₃-C₁₂)-cycloalkyl, —S—(C₁-C₁₂)-alkyl, —S—(C₃-C₁₂)-cycloalkyl, —COO—(C₁-C₁₂)-alkyl, —COO—(C₃-C₁₂)-cycloalkyl, —CONH—(C₁-C₁₂)-alkyl, —CONH—(C₃-C₁₂)-cycloalkyl, —CO—(C₁-C₁₂)-alkyl, —CO—(C₃-C₁₂)-cycloalkyl, —N—[(C₁-C₁₂)-alkyl]₂, —(C₆-C₂₀)-aryl, —(C₆-C₂₀)-aryl-(C₁-C₁₂)-alkyl, —(C₆-C₂₀)-aryl-O—(C₁-C₁₂)-alkyl, —(C₃-C₂₀)-heteroaryl, —(C₃-C₂₀)-heteroaryl-(C₁-C₁₂)-alkyl, —(C₃-C₂₀)-heteroaryl-O—(C₁-C₁₂)-alkyl, —COOH, —OH, —SH, —SO₃H, —CN, —NH₂, or a halogen;

X, Y, and Z are independently selected from —O, —NH, —S, —N—(C₁-C₃₀)-alkyl, or —(C₁-C₃₀)-aryl;

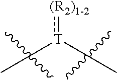

is —CH((C₁-C₃₀)-alkyl)), —(C═O)—, —CH(OH), —SO₂—, —SO—, or —CH(SOCH₃)—; and

R₄ is selected from —(C₁-C₁₂)-alkyl, —(C₃-C₁₂)-cycloalkyl, —(C₃-C₁₂)-heterocycloalkyl, —O—(C₁-C₁₂)-alkyl, —O—(C₁-C₁₂)-alkyl-(C₆-C₂₀)-aryl, —O—(C₃-C₁₂)-cycloalkyl, —S—(C₁-C₁₂)-alkyl, —S—(C₃-C₁₂)-cycloalkyl, —COO—(C₁-C₁₂)-alkyl, —COO—(C₃-C₁₂)-cycloalkyl, —CONH—(C₁-C₁₂)-alkyl, —CONH—(C₃-C₁₂)-cycloalkyl, —CO—(C₁-C₁₂)-alkyl, —CO—(C₃-C₁₂)-cycloalkyl, —N—[(C₁-C₁₂)-alkyl]₂, —(C₆-C₂₀)-aryl, —(C₆-C₂₀)-aryl-(C₁-C₁₂)-alkyl, —(C₆-C₂₀)-aryl-O—(C₁-C₁₂)-alkyl, —(C₃-C₂₀)-heteroaryl, —(C₃-C₂₀)-heteroaryl-(C₁-C₁₂)-alkyl, —(C₃-C₂₀)-heteroaryl-O—(C₁-C₁₂)-alkyl, —COOH, —OH, —SH, —SO₃H, —CN, —NH₂, or a halogen.

Still another embodiment provides the compound of Formula IV:

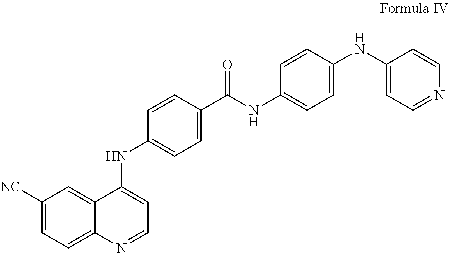 [SMILES: N#Cc1ccc2nccc(Nc3ccc(C(=O)Nc4ccc(Nc5ccncc5)cc4)cc3)c2c1]

or a pharmaceutically acceptable enantiomer, salt, or solvate thereof.

The compound of Formula IV, also referred to as mJJ64A, and enantiomers, polymorphs, pharmaceutically acceptable salts, and derivatives thereof can be used to induce, promote, or increase Akt3 bioactivity in immune cells.

In some embodiments, the Atk3 activator is a derivative of Formula I, Formula II, Formula III, or Formula IV. The term “derivative” or “derivatized” as used herein includes one or more chemical modifications of Formula I, Formula II, Formula III, or Formula IV, or an enantiomer, polymorph, or pharmaceutically acceptable salt thereof. That is, a “derivative” may be a functional equivalent of Formula I, Formula II, Formula III, or Formula IV which is capable of inducing the improved pharmacological functional activity and/or behavioral response in a given subject. Illustrative of such chemical modifications would be replacement of hydrogen by a halo group, an alkyl group, an acyl group or an amino group.

The chemical modification of Formula I, Formula II, Formula III, or Formula IV, or an enantiomer, polymorph, or pharmaceutically acceptable salt thereof, may either enhance or reduce hydrogen bonding interaction, charge interaction, hydrophobic interaction, Van der Waals interaction, or dipole-dipole interaction between the compound and its target.

In some embodiments, the compound of Formula I, Formula II, Formula III, or Formula IV may act as a model (for example, a template) for the development of other derivative compounds which are a functional equivalents of the compound and which are capable of inducing the improved pharmacological functional activity and/or effect and/or behavioral response in a given subject.

The compound of Formula I, Formula II, Formula III, or Formula IV may be a racemic compound and/or optically active isomers thereof. In this regard, some of the compounds can have asymmetric carbon atoms, and therefore, can exist either as racemic mixtures or as individual optical isomers (for example, enantiomers). Compounds described herein that contain a chiral center include all possible stereoisomers of the compound, including compositions including the racemic mixture of the two enantiomers, as well as compositions including each enantiomer individually, substantially free of the other enantiomer. Thus, for example, contemplated herein is a composition including the S enantiomer of a compound substantially free of the R enantiomer, or the R enantiomer substantially free of the S enantiomer. If the named compound includes more than one chiral center, the scope of the present disclosure also includes compositions including mixtures of varying proportions between the diastereomers, as well as compositions including one or more diastereomers substantially free of one or more of the other diastereomers. By “substantially free” it is meant that the composition includes less than about 25%, 15%, 10%, 8%, 5%, 3%, or less than about 1% of the minor enantiomer or diastereomer(s).

B. Akt3 Inhibitor Compounds

Compositions and methods of selectively inhibiting Akt3 are disclosed herein. Exemplary Akt3 inhibitors are described in U.S. Patent Publication Nos. US2017/0202956 and 2017/0202829 (each incorporated by reference herein in its entirety) and are described below.

It has been discovered that 4-[(6-nitroquinolin-4-yl)amino]-N-[4-(pyridin-4-ylamino)phenyl]benzamide selectively inhibits Akt3 activity. 4-[(6-nitroquinolin-4-yl)amino]-N-[4-(pyridin-4-ylamino)phenyl]benzamide has CAS No. 50440-30-7 and the following chemical structure:

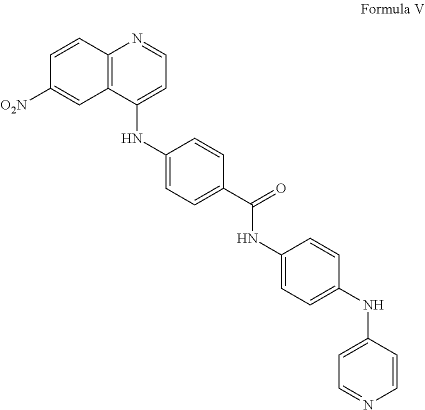 [SMILES: O=C(Nc1ccc(Nc2ccncc2)cc1)c1ccc(Nc2ccnc3ccc([N+](=O)[O-])cc23)cc1]

Other exemplary compounds for selectively inhibiting Akt3 include the following:

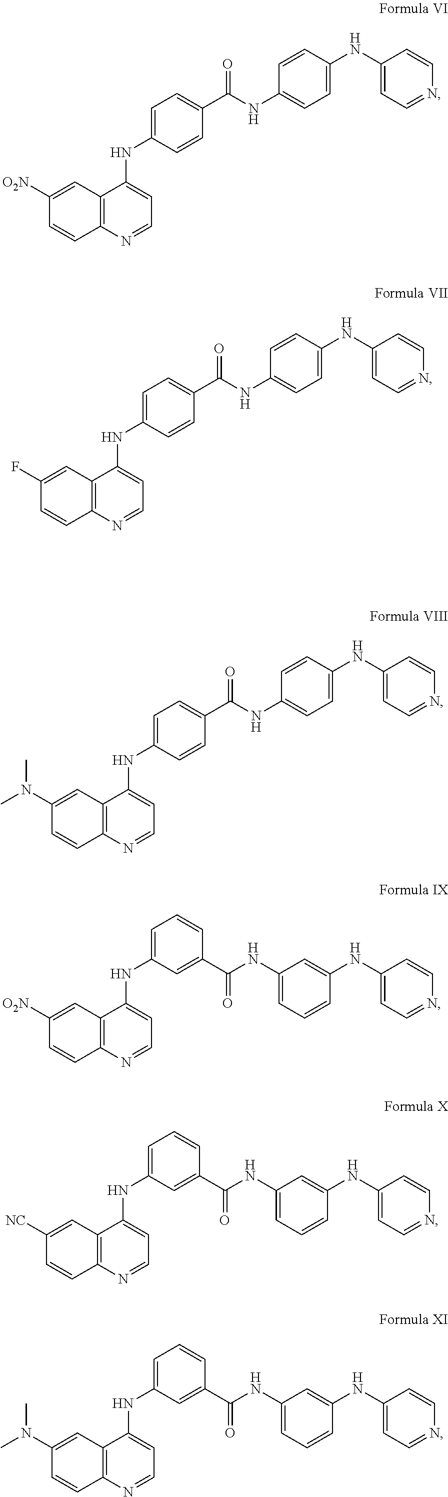 [SMILES: O=C(Nc1ccc(Nc2ccncc2)cc1)c1ccc(Nc2ccnc3ccc([N+](=O)[O-])cc23)cc1 ; O=C(Nc1ccc(Nc2ccncc2)cc1)c1ccc(Nc2ccnc3ccc(F)cc23)cc1 ; CN(C)c1ccc2nccc(Nc3ccc(C(=O)Nc4ccc(Nc5ccncc5)cc4)cc3)c2c1 ; O=C(Nc1cccc(Nc2ccncc2)c1)c1cccc(Nc2ccnc3ccc([N+](=O)[O-])cc23)c1 ; N#Cc1ccc2nccc(Nc3cccc(C(=O)Nc4cccc(Nc5ccncc5)c4)c3)c2c1 ; CN(C)c1ccc2nccc(Nc3cccc(C(=O)Nc4cccc(Nc5ccncc5)c4)c3)c2c1]

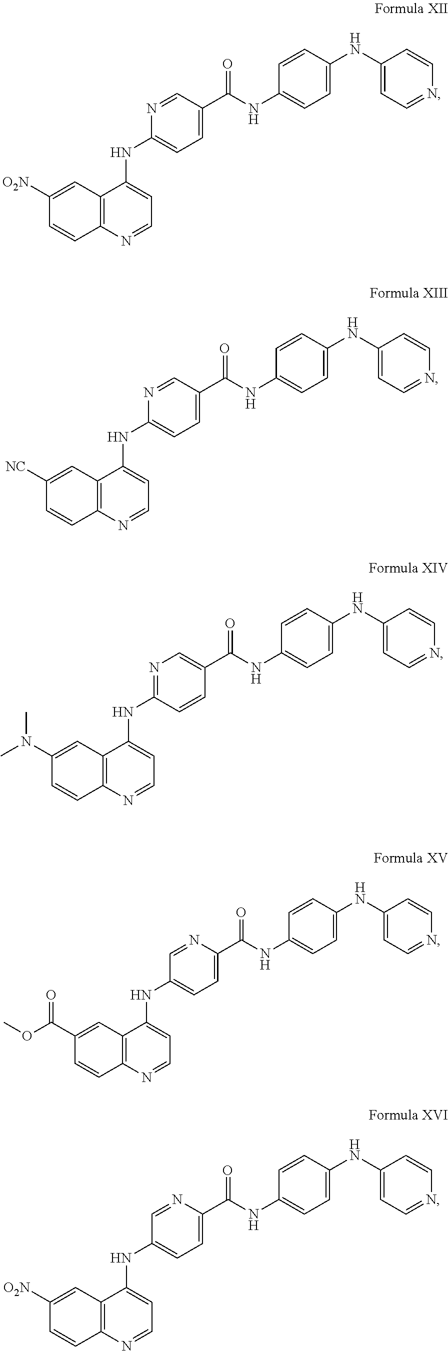 [SMILES: O=C(Nc1ccc(Nc2ccncc2)cc1)c1ccc(Nc2ccnc3ccc([N+](=O)[O-])cc23)nc1 ; N#Cc1ccc2nccc(Nc3ccc(C(=O)Nc4ccc(Nc5ccncc5)cc4)cn3)c2c1 ; CN(C)c1ccc2nccc(Nc3ccc(C(=O)Nc4ccc(Nc5ccncc5)cc4)cn3)c2c1 ; COC(=O)c1ccc2nccc(Nc3ccc(C(=O)Nc4ccc(Nc5ccncc5)cc4)nc3)c2c1 ; O=C(Nc1ccc(Nc2ccncc2)cc1)c1ccc(Nc2ccnc3ccc([N+](=O)[O-])cc23)cn1]

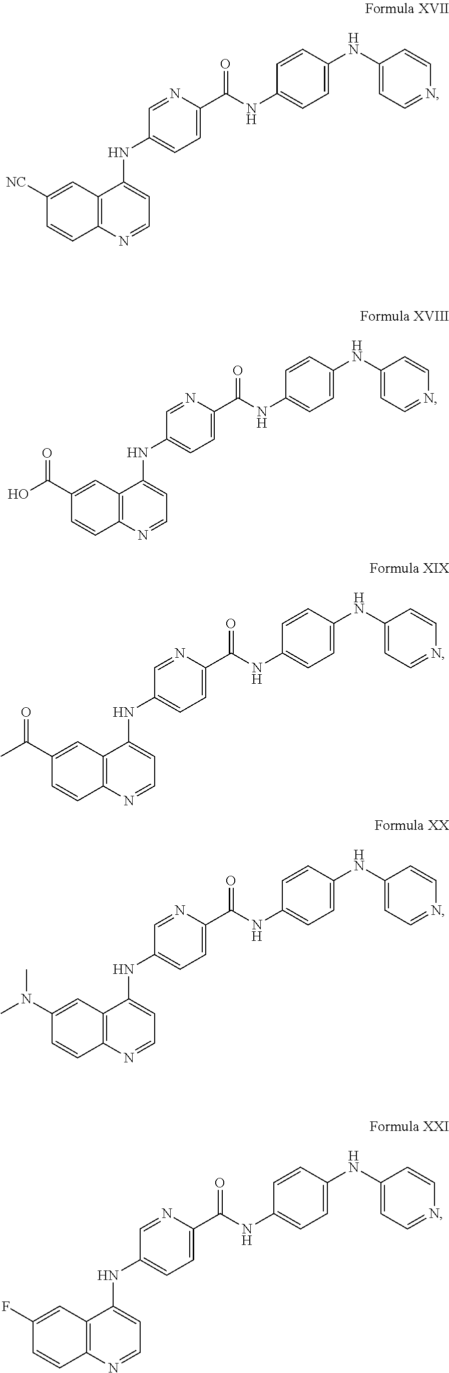 [SMILES: N#Cc1ccc2nccc(Nc3ccc(C(=O)Nc4ccc(Nc5ccncc5)cc4)nc3)c2c1 ; O=C(O)c1ccc2nccc(Nc3ccc(C(=O)Nc4ccc(Nc5ccncc5)cc4)nc3)c2c1 ; CC(=O)c1ccc2nccc(Nc3ccc(C(=O)Nc4ccc(Nc5ccncc5)cc4)nc3)c2c1 ; CN(C)c1ccc2nccc(Nc3ccc(C(=O)Nc4ccc(Nc5ccncc5)cc4)nc3)c2c1 ; O=C(Nc1ccc(Nc2ccncc2)cc1)c1ccc(Nc2ccnc3ccc(F)cc23)cn1]

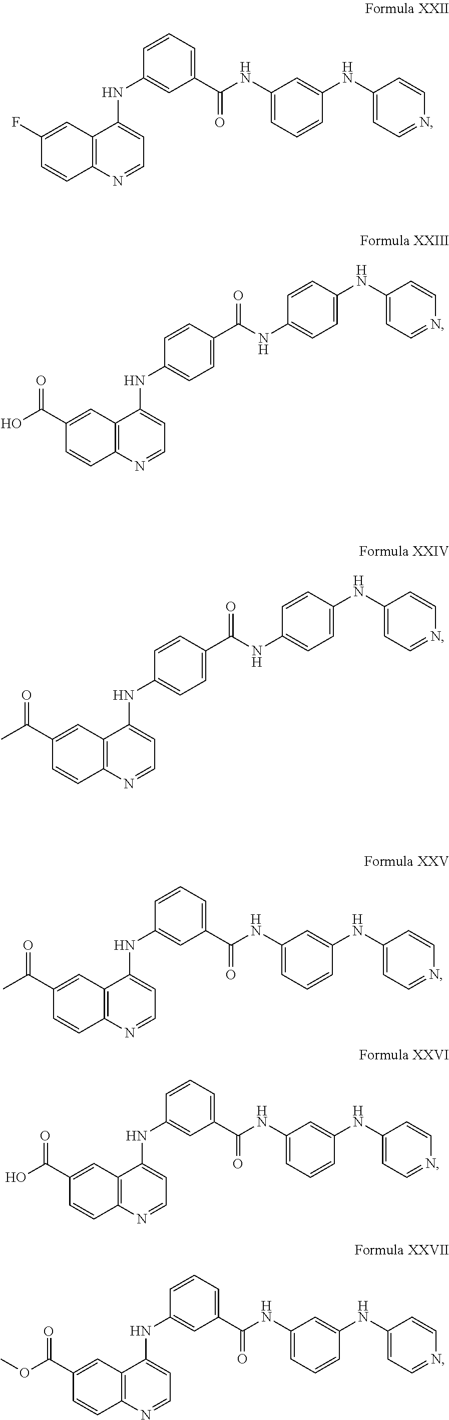 [SMILES: O=C(Nc1cccc(Nc2ccncc2)c1)c1cccc(Nc2ccnc3ccc(F)cc23)c1 ; O=C(O)c1ccc2nccc(Nc3ccc(C(=O)Nc4ccc(Nc5ccncc5)cc4)cc3)c2c1 ; CC(=O)c1ccc2nccc(Nc3ccc(C(=O)Nc4ccc(Nc5ccncc5)cc4)cc3)c2c1 ; CC(=O)c1ccc2nccc(Nc3cccc(C(=O)Nc4cccc(Nc5ccncc5)c4)c3)c2c1 ; O=C(O)c1ccc2nccc(Nc3cccc(C(=O)Nc4cccc(Nc5ccncc5)c4)c3)c2c1 ; COC(=O)c1ccc2nccc(Nc3cccc(C(=O)Nc4cccc(Nc5ccncc5)c4)c3)c2c1]

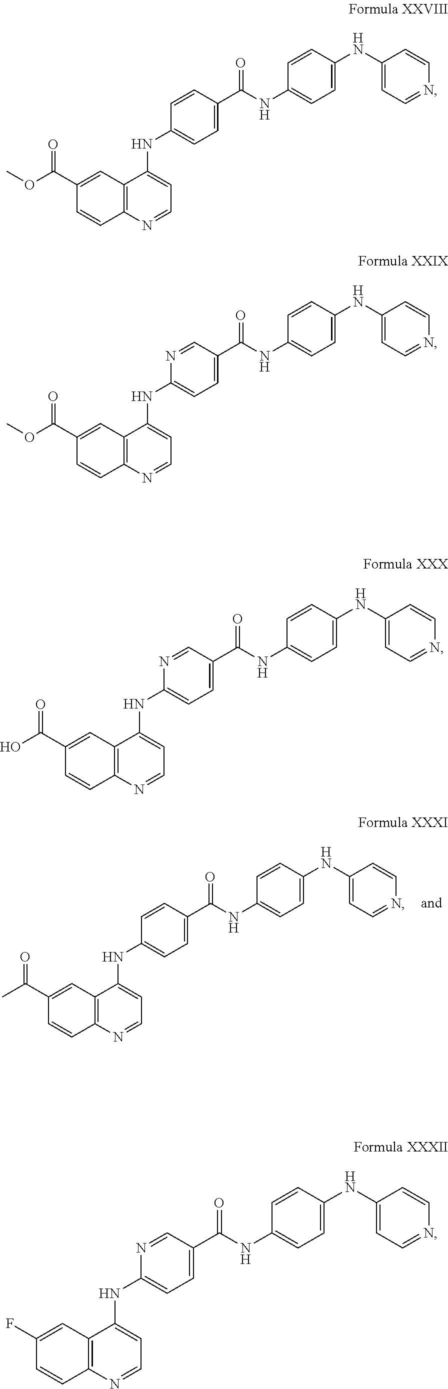 [SMILES: COC(=O)c1ccc2nccc(Nc3ccc(C(=O)Nc4ccc(Nc5ccncc5)cc4)cc3)c2c1 ; COC(=O)c1ccc2nccc(Nc3ccc(C(=O)Nc4ccc(Nc5ccncc5)cc4)cn3)c2c1 ; O=C(O)c1ccc2nccc(Nc3ccc(C(=O)Nc4ccc(Nc5ccncc5)cc4)cn3)c2c1 ; CC(=O)c1ccc2nccc(Nc3ccc(C(=O)Nc4ccc(Nc5ccncc5)cc4)cc3)c2c1 ; O=C(Nc1ccc(Nc2ccncc2)cc1)c1ccc(Nc2ccnc3ccc(F)cc23)nc1]

and enantiomers, polymorphs, pharmaceutically acceptable salts, and derivatives thereof.

In some embodiments, the Akt3 inhibitor is a derivative of any one of the disclosed compounds. The term “derivative” or “derivatized” as used herein includes one or more chemical modifications of any one of the disclosed compounds, an enantiomer, polymorph, or pharmaceutically acceptable salt thereof. That is, a “derivative” may be a functional equivalent of any one of the disclosed compounds, which is capable of inducing the improved pharmacological functional activity and/or behavioral response in a given subject. Illustrative of such chemical modifications would be replacement of hydrogen by a halo group, an alkyl group, an acyl group, or an amino group.

The chemical modification of any one of the disclosed compounds, or an enantiomer, polymorph, or pharmaceutically acceptable salt thereof, may either enhance or reduce hydrogen bonding interaction, charge interaction, hydrophobic interaction, Van der Waals interaction or dipole interaction between the compound and its target.

In some embodiments, the compound of any one of the disclosed compounds may act as a model (for example, a template) for the development of other derivative compounds which are a functional equivalents of the compound and which are capable of inducing the improved pharmacological functional activity and/or effect and/or behavioral response in a given subject.

The disclosed compounds may be racemic compounds and/or optically active isomers thereof. In this regard, some of the compounds can have asymmetric carbon atoms, and therefore, can exist either as racemic mixtures or as individual optical isomers (enantiomers). Compounds described herein that contain a chiral center include all possible stereoisomers of the compound, including compositions including the racemic mixture of the two enantiomers, as well as compositions including each enantiomer individually, substantially free of the other enantiomer. Thus, for example, contemplated herein is a composition including the S enantiomer of a compound substantially free of the R enantiomer, or the R enantiomer substantially free of the S enantiomer. If the named compound includes more than one chiral center, the scope of the present disclosure also includes compositions including mixtures of varying proportions between the diastereomers, as well as compositions including one or more diastereomers substantially free of one or more of the other diastereomers. By “substantially free” it is meant that the composition includes less than about 25%, 15%, 10%, 8%, 5%, 3%, or less than about 1% of the minor enantiomer or diastereomer(s).

The disclosed compounds selectively modulate Akt3 compared to Akt1 and Akt2. In certain embodiments, any one of the disclosed compounds do not modulate Akt1 and Akt2 to a statistically significant degree. In other embodiments, modulation of Akt3 by the disclosed compounds is about 5, 10, 15, 50, 100, 1000, or 5000-fold greater than their modulation of Akt1 and Akt2.

C. Immunomodulatory Agents or Binding Moieties

Immunomodulatory agents or binding moieties including agonists and antagonists of AKT3 are provided. An agonist of AKT3 typically induces, promotes, or enhances AKT3 mediated signaling. An antagonist of AKT3 typically inhibits, reduces, or blocks AKT3 mediated signaling. The disclosed compositions and methods can be used to modulate AKT3 and/or counter-receptor signaling on, for example, immune cells including, but not limited to, monocytes, Tregs, tumor-associated macrophages (TAMs), myeloid derived suppressor cells (MDSC), T cells, Th2 cells, myeloid cells including antigen-presenting cells (e.g., monocyte, macrophage, or dendritic cells), T cells, NK cells, or a combination thereof. In some embodiments, the compositions are specifically targeted to one or more cell types. In some embodiments, the disclosed compositions can be used on tumor cells.

In some embodiments, the anti-AKT3 agonists induce, promote, or enhance AKT3 mediated signaling through a known ligand or unknown counter-receptor through AKT3 interaction with said known or unknown counter-receptor. For example, in some embodiments, the AKT3 agonist binds to, induces, promotes, or creates a conformational change, or otherwise promotes AKT3 mediated signal transduction.

In some embodiments, the anti-AKT3 antagonists inhibit, reduce, block, or otherwise disrupt signaling through a known or unknown counter-receptor through blockade of AKT3 interaction with said known or unknown counter-receptor. For example, in some embodiments, the AKT3 antagonist binds to, inhibits, blocks, creates a conformational change, or otherwise interferes with AKT3 mediated signal transduction.

1. Antibodies

In one embodiment the immunomodulatory agent or binding moiety is an antibody. Suitable antibodies can be prepared by one of skill in the art. Nucleic acid and polypeptide sequences for AKT3 are known in the art and exemplary sequences are provided above. The sequences can be used, as discussed in more detail below, by one of skill in the art to prepare an antibody or antigen binding fragment thereof specific for AKT3. The antibody or antigen binding fragment, therefore, can be an agonist or antagonist of AKT3-mediated signaling.

The activity of an antibody or antigen binding fragment thereof that is specific for AKT3 can be determined using functional assays that are known in the art, and include the assays discussed below. Typically, the assays include determining if the antibody or antigen binding fragment thereof increases (i.e., agonist) or decreases (i.e., antagonist) signaling through AKT3.

In some embodiments, the disclosed antibodies and antigen binding fragments thereof immunospecifically bind to human or mouse AKT3. In some embodiments, the antibody binds to an extracellular domain of human or mouse AKT3.

To prepare an antibody or antigen binding fragment thereof that specifically binds to AKT3 purified proteins, polypeptides, fragments, fusions, or epitopes to AKT3 or polypeptides expressed from nucleic acid sequences thereof, can be used. The antibodies or antigen binding fragments thereof can be prepared using any suitable methods known in the art, such as those discussed in more detail below.

a. Human and Humanized Antibodies

In some embodiments, the antibodies are humanized antibodies. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge.

Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementarity determining region (CDR) of the recipient antibody are replaced by residues from a CDR of a non-human species (donor antibody), such as mouse, rat, or rabbit having the desired specificity, affinity, and capacity. In some instances, Fv framework residues of the human immunoglobulin are replaced by corresponding non-human residues. Humanized antibodies may also contain residues that are found neither in the recipient antibody nor in the imported CDR or framework sequences. In general, the humanized antibody will contain substantially all of at least one, and typically two, variable domains, in which all or substantially all of the CDR regions correspond to those of a non-human immunoglobulin, and all or substantially all of the FR regions are those of a human immunoglobulin consensus sequence. The humanized antibody optimally also will contain at least a portion of an immunoglobulin constant region (Fc), typically that of a human immunoglobulin.

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule. Humanization can be essentially performed by substituting rodent CDRs or CDR sequences for the corresponding sequences of a human antibody. Accordingly, a humanized form of a nonhuman antibody (or a fragment thereof) is a chimeric antibody or fragment, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody. Another method uses a particular framework derived from the consensus sequence of all human antibodies of a particular subgroup of light or heavy chains. The same framework may be used for several different humanized antibodies.

It is further important that antibodies be humanized with retention of high affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding.

The antibody can be bound to a substrate or labeled with a detectable moiety or be both bound and labeled. The detectable moieties contemplated with the present compositions include fluorescent, enzymatic, and radioactive markers.

b. Single-Chain Antibodies

In some embodiments, the antibodies are single-chain antibodies. Methods for the production of single-chain antibodies are well known to those of skill in the art. A single chain antibody is created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation. These Fvs lack the constant regions (Fc) present in the heavy and light chains of the native antibody.

c. Monovalent Antibodies

In some embodiments, the antibodies are monovalent antibodies. In vitro methods are also suitable for preparing monovalent antibodies. Digestion of antibodies to produce fragments thereof, particularly, Fab fragments, can be accomplished using routine techniques known in the art. For instance, digestion can be performed using papain. Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

d. Hybrid Antibodies

In some embodiments, the antibodies are hybrid antibodies. In hybrid antibodies, one heavy and light chain pair is homologous to that found in an antibody raised against one epitope, while the other heavy and light chain pair is homologous to a pair found in an antibody raised against another epitope. This results in the property of multi-functional valency, i.e., ability to bind at least two different epitopes simultaneously. Such hybrids can be formed by fusion of hybridomas producing the respective component antibodies, or by recombinant techniques. Such hybrids may, of course, also be formed using chimeric chains.

e. Conjugates or Fusions of Antibody Fragments

In some embodiments, the antibodies are conjugates or fusions of antibody fragments. The targeting function of the antibody can be used therapeutically by coupling the antibody or a fragment thereof with a therapeutic agent. Such coupling of the antibody or fragment (e.g., at least a portion of an immunoglobulin constant region (Fc)) with the therapeutic agent can be achieved by making an immunoconjugate or by making a fusion protein, comprising the antibody or antibody fragment and the therapeutic agent.

Such coupling of the antibody or fragment with the therapeutic agent can be achieved by making an immunoconjugate or by making a fusion protein, or by linking the antibody or fragment to a nucleic acid, such as an siRNA, comprising the antibody or antibody fragment and the therapeutic agent.

In some embodiments, the antibody is modified to alter its half-life. In some embodiments, it is desirable to increase the half-life of the antibody so that it is present in the circulation or at the site of treatment for longer periods of time. For example, it may be desirable to maintain titers of the antibody in the circulation or in the location to be treated for extended periods of time. Antibodies can be engineered with Fc variants that extend half-life, e.g., using Xtend™ antibody half-life prolongation technology (Xencor, Monrovia, Calif.). In other embodiments, the half-life of the anti-DNA antibody is decreased to reduce potential side effects. The conjugates disclosed can be used for modifying a given biological response. The drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin.

2. Proteins and Polypeptides

a. Protein and Polypeptide Compositions

The immunomodulatory or binding agent can be a AKT3 protein, polypeptide, or fusion protein. For example, the immunomodulatory agent or binding moiety can be an isolated or recombinant protein or polypeptide, or functional fragment, variant, or fusion protein thereof of AKT3.

The AKT3 protein or polypeptide, or functional fragment, variant, or fusion protein thereof can be an agonist or an antagonist. For example, in some embodiments an antagonist of AKT3 is a AKT3 polypeptide or a fragment or fusion protein thereof that binds to a ligand of AKT3. The polypeptide can be a soluble fragment, for example the extracellular domain of AKT3, or a functional fragment thereof, or a fusion protein thereof. In some embodiments, a soluble ligand of AKT3 may serve as an antagonist, decreasing AKT3 mediated signal transduction.

The activity of a protein or polypeptide of AKT3, or any fragment, variant, or fusion protein thereof can be determined using functional assays that are known in the art, and include the assays discussed below. Typically, the assays include determining if the protein, polypeptide or fragment, variant or fusion protein thereof increases (i.e., agonist) or decreases (i.e., antagonist) signaling through the AKT3 receptor. In some embodiments, the assay includes determining if the protein, polypeptide or fragment, variant, or fusion protein thereof increases (i.e., agonist) or decreases (i.e., antagonist) the immune response associated with AKT3. Typically, the assays include determining if the protein, polypeptide or fragment, variant, or fusion protein thereof increases (i.e., agonist) or decreases (i.e., antagonist) signaling through AKT3. In some embodiments, the assay includes determining if the protein, polypeptide or fragment, variant, or fusion protein thereof decreases (i.e., agonist) or increases (i.e., antagonist) an immune response regulated by AKT3. In some embodiments, the assay includes determining if the protein, polypeptide or fragment, variant, or fusion protein thereof increases (i.e., antagonist) the apoptosis and differentiation of acute myeloid leukemia (AML) cells and acute lymphoblastic leukemia (ALL) cells resulting in reduced self-renewal capacity of AML and ALL stem cells.

Nucleic acid and polypeptide sequences for AKT3 are known in the art and exemplary protein and peptide sequences are provided above. The sequences can be used, as discussed in more detail below, by one of skill in the art to prepare any protein or polypeptide of AKT3, or any fragment, variant, or fusion protein thereof. Generally, the proteins, polypeptides, fragments, variants, and fusions thereof of AKT3 are expressed from nucleic acids that include sequences that encode a signal sequence. The signal sequence is generally cleaved from the immature polypeptide to produce the mature polypeptide lacking the signal sequence. The signal sequence can be replaced by the signal sequence of another polypeptide using standard molecular biology techniques to affect the expression levels, secretion, solubility, or other property of the polypeptide AKT3 proteins with and without a signal sequence. It is understood that in some cases, the mature protein as it is known or described in the art, i.e., the protein sequence without the signal sequence is a putative mature protein. During normal cell expression, a signal sequence can be removed by a cellular peptidase to yield a mature protein. The sequence of the mature protein can be determined or confirmed using methods that are known in the art.

i. Fragments

As used herein, a fragment of AKT3 refers to any subset of the polypeptide that is at least one amino acid shorter than full length protein. Useful fragments include those that retain the ability to bind to their natural ligand or ligands. A polypeptide that is a fragment of any full-length AKT3 typically has at least about 20 percent, 30 percent, 40 percent, 50 percent, 60 percent, 70 percent, 80 percent, 90 percent, 95 percent, 98 percent, 99 percent, 100 percent, or even more than 100 percent of the ability to bind its natural ligand respectively as compared to the full-length protein.

Fragments of AKT3 include cell free fragments. Cell free polypeptides can be fragments of full-length, transmembrane, polypeptides that may be shed, secreted or otherwise extracted from the producing cells. Cell free fragments of polypeptides can include some or all of the extracellular domain of the polypeptide, and lack some or all of the intracellular and/or transmembrane domains of the full-length protein. In one embodiment, polypeptide fragments include the entire extracellular domain of the full-length protein. In other embodiments, the cell free fragments of the polypeptides include fragments of the extracellular domain that retain biological activity of full-length protein. The extracellular domain can include 1, 2, 3, 4, or 5 contiguous amino acids from the transmembrane domain, and/or 1, 2, 3, 4, or 5 contiguous amino acids from the signal sequence. Alternatively, the extracellular domain can have 1, 2, 3, 4, 5 or more amino acids removed from the C-terminus, N-terminus, or both. In some embodiments the extracellular domain is the only functional domain of the fragment (e.g., the ligand binding domain).

ii. Variants

Variants of AKT3, and fragments thereof are also provided. In some embodiments, the variant is at least about 50, 60, 70, 80, 85, 90, 95, 96, 97, 98, or 99 percent identical to any one of SEQ ID NO:1 or 2. Useful variants include those that increase biological activity, as indicated by any of the assays described herein, or that increase half-life or stability of the protein. The protein and polypeptides of AKT3, and fragments, variants, and fusion proteins thereof can be engineered to increase biological activity. For example, in some embodiments, an AKT3 polypeptide, protein, or fragment, variant, or fusion thereof has been modified with at least one amino acid substitution, deletion, or insertion that increases a function thereof.

Finally, variant polypeptides can be engineered to have an increased half-life relative to wild type. These variants typically are modified to resist enzymatic degradation. Exemplary modifications include modified amino acid residues and modified peptide bonds that resist enzymatic degradation. Various modifications to achieve this are known in the art. The variants can be modified to adjust for effects of affinity for the receptor on the half-life of proteins, polypeptides, fragments, or fusions thereof at serum and endosomal pH.

iii. Fusion Proteins

Fusion polypeptides have a first fusion partner including all or a part of a human or mouse AKT3 polypeptide fused to a second polypeptide directly or via a linker peptide sequence that is fused to the second polypeptide. In one embodiment, the ECD of human or mouse AKT3 or a fragment thereof is fused to a second polypeptide. The fusion proteins optionally contain a domain that functions to dimerize or multimerize two or more fusion proteins. The peptide/polypeptide linker domain can either be a separate domain, or alternatively can be contained within one of the other domains (first polypeptide or second polypeptide) of the fusion protein. Similarly, the domain that functions to dimerize or multimerize the fusion proteins can either be a separate domain, or alternatively can be contained within one of the other domains (first polypeptide, second polypeptide, or peptide/polypeptide linker domain) of the fusion protein. In one embodiment, the dimerization/multimerization domain and the peptide/polypeptide linker domain are the same.

Fusion proteins disclosed herein are of Formula A:

N—P1-P2-P3-C

wherein “N” represents the N-terminus of the fusion protein and “C” represents the C-terminus of the fusion protein. In some embodiments, “P1” is a polypeptide or protein of AKT3 or fragment or variant thereof, “P2” is an optional peptide/polypeptide linker domain, and “P3” is a second polypeptide. Alternatively, P3 may be a polypeptide or protein of AKT3, or fragment or variant thereof and P1 may be a second polypeptide. In some embodiments, the AKT3 polypeptide is the extracellular domain.

Dimerization or multimerization can occur between or among two or more fusion proteins through dimerization or multimerization domains. Alternatively, dimerization or multimerization of fusion proteins can occur by chemical crosslinking. The dimers or multimers that are formed can be homodimeric/homomultimeric or heterodimeric/heteromultimeric.

In some embodiments, the fusion protein includes the extracellular domain of AKT3, or a fragment or variant thereof, fused to an Ig Fc region. Recombinant Ig fusion proteins can be prepared by fusing the coding region of the extracellular domain or a fragment or variant thereof to the Fc region of human IgG1, IgG2, IgG3, or IgG4 or mouse IgG2a, or other suitable Ig domain, as described previously (Chapoval, et al., Methods Mol. Med., 45:247-255 (2000); incorporated herein by reference in its entirety).

iv. Polypeptide Modifications

The polypeptides and fusion proteins may be modified by chemical moieties that may be present in polypeptides in a normal cellular environment, for example, phosphorylation, methylation, amidation, sulfation, acylation, glycosylation, sumoylation, and ubiquitylation. Fusion proteins may also be modified with a label capable of providing a detectable signal, either directly or indirectly, including, but not limited to, radioisotopes and fluorescent compounds.

The polypeptides and fusion proteins may also be modified by chemical moieties that are not normally added to polypeptides in a cellular environment. For example, the disclosed fusion proteins may also be modified by covalent attachment of polymer chains, including, but not limited to, polyethylene glycol polymer (PEG) chains (i.e., pegylation). Conjugation of macromolecules to PEG has emerged recently as an effective strategy to alter the pharmacokinetic (PK) profiles of a variety of drugs, and thereby to improve their therapeutic potential. PEG conjugation increases retention of drugs in the circulation by protecting against enzymatic digestion, slowing filtration by the kidneys, and reducing the generation of neutralizing antibodies. In addition, PEG conjugates can be used to allow multimerization of the fusion proteins.

Modifications may be introduced into the molecule by reacting targeted amino acid residues of the polypeptide with an organic derivatizing agent that is capable of reacting with selected side chains or terminal residues. Another modification is cyclization of the protein.

Examples of chemical derivatives of the polypeptides include lysinyl and amino terminal residues derivatized with succinic or other carboxylic acid anhydrides. Derivatization with a cyclic carboxylic anhydride has the effect of reversing the charge of the lysinyl residues. Other suitable reagents for derivatizing amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate. Carboxyl side groups, aspartyl or glutamyl, may be selectively modified by reaction with carbodiimides (R—N═C═N—R′) such as 1-cyclohexyl-3-(2-morpholinyl-(4-ethyl)carbodiimide or 1-ethyl-3-(4-azonia-4,4-dimethylpentyl) carbodiimide. Furthermore, aspartyl and glutamyl residues can be converted to asparaginyl and glutaminyl residues by reaction with ammonia. Fusion proteins may also include one or more D-amino acids that are substituted for one or more L-amino acids.

v. Modified Binding Properties

Binding properties of the proteins, polypeptides, fragments, variants, and fusions thereof are relevant to the dose and dose regimen to be administered. In one embodiment the disclosed proteins, polypeptides, fragments, variants, and fusions thereof have binding properties to AKT3 or an AKT3 ligand that demonstrate a higher term, or higher percentage, of occupancy of a binding site (e.g., on the ligand) relative to other receptor molecules that bind thereto. In other embodiments, the disclosed proteins, polypeptides, fragments, variants, and fusions thereof have reduced binding affinity to AKT3 relative to wild type protein.

In some embodiments the proteins, polypeptides, fragments, variants, and fusions thereof have a relatively high affinity for AKT3 and may therefore have a relatively slow off rate. In other embodiments, the proteins polypeptides, fragments, variants, and fusions thereof are administered intermittently over a period of days, weeks, or months to dampen immune responses which are allowed to recover prior to the next administration, which may serve to alter the immune response without completely turning the immune response on or off and may avoid long term side effects.

3. Isolated Nucleic Acid Molecules

Isolated nucleic acid sequences encoding the AKT3 proteins, polypeptides, fragments, variants, and fusions thereof are disclosed herein. As used herein, “isolated nucleic acid” refers to a nucleic acid that is separated from other nucleic acid molecules that are present in a mammalian genome, including nucleic acids that normally flank one or both sides of the nucleic acid in a mammalian genome. The term “isolated” as used herein with respect to nucleic acids also includes the combination with any non-naturally occurring nucleic acid sequence, since such non-naturally occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid, such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

Nucleic acids encoding the proteins, polypeptides, fragments, variants and fusions thereof may be optimized for expression in the expression host of choice. Codons may be substituted with alternative codons encoding the same amino acid to account for differences in codon usage between the mammal from which the nucleic acid sequence is derived and the expression host. In this manner, the nucleic acids may be synthesized using expression host-preferred codons.

Nucleic acids can be in sense or antisense orientation or can be complementary to a reference sequence encoding a polypeptide or protein of AKT3. Nucleic acids can be DNA, RNA, or nucleic acid analogs. Nucleic acid analogs can be modified at the base moiety, sugar moiety, or phosphate backbone. Such modifications can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorg. Med. Chem. 4:5-23 (each incorporated by reference herein in its entirety). In addition, the deoxyphosphate backbone can be replaced with, for example, a phosphorothioate or phosphorodithioate backbone, a phosphoroamidite, or an alkyl phosphotriester backbone.

Nucleic acids encoding polypeptides can be administered to subjects in need thereof. Nucleic delivery involves introduction of “foreign” nucleic acids into a cell and ultimately, into a live animal. Compositions and methods for delivering nucleic acids to a subject are known in the art (see Understanding Gene Therapy, Lemoine, N. R., ed., BIOS Scientific Publishers, Oxford, 2008; incorporated herein by reference in its entirety).

4. Vectors and Host Cells

Vectors encoding the proteins, polypeptides, fragments, variants, and fusions thereof are also provided. Nucleic acids, such as those described above, can be inserted into vectors for expression in cells. As used herein, a “vector” is a replicon, such as a plasmid, phage, virus, or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen Life Technologies (Carlsbad, Calif.).

An expression vector can include a tag sequence. Tag sequences are typically expressed as a fusion with the encoded polypeptide. Such tags can be inserted anywhere within the polypeptide including at either the carboxyl or amino terminus. Examples of useful tags include, but are not limited to, green fluorescent protein (GFP), glutathione S-transferase (GST), polyhistidine, c-myc, hemagglutinin, Flag™ tag (Kodak, New Haven, Conn.), maltose E binding protein, and protein A. In one embodiment, a nucleic acid molecule encoding one of the disclosed polypeptides is present in a vector containing nucleic acids that encode one or more domains of an Ig heavy chain constant region, for example, having an amino acid sequence corresponding to the hinge, CH2, and CH3 regions of a human immunoglobulin Cy1 chain.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell such as a CHO cell) can be used to, for example, produce the proteins, polypeptides, fragments, variants, and fusions thereof described herein.

The vectors described can be used to express the proteins, polypeptides, fragments, variants, and fusions thereof in cells. An exemplary vector includes, but is not limited to, an adenoviral vector. One approach includes nucleic acid transfer into primary cells in culture followed by autologous transplantation of the ex vivo transformed cells into the host, either systemically or into a particular organ or tissue. Ex vivo methods can include, for example, the steps of harvesting cells from a subject, culturing the cells, transducing them with an expression vector, and maintaining the cells under conditions suitable for expression of the encoded polypeptides. These methods are known in the art of molecular biology. The transduction step can be accomplished by any standard means used for ex vivo gene therapy, including, for example, calcium phosphate, lipofection, electroporation, viral infection, and biolistic gene transfer. Alternatively, liposomes or polymeric microparticles can be used. Cells that have been successfully transduced then can be selected, for example, for expression of the coding sequence or of a drug resistance gene. The cells then can be lethally irradiated (if desired) and injected or implanted into the subject. In one embodiment, expression vectors containing nucleic acids encoding fusion proteins are transfected into cells that are administered to a subject in need thereof.

In vivo nucleic acid therapy can be accomplished by direct transfer of a functionally active DNA into mammalian somatic tissue or organ in vivo. For example, nucleic acids encoding polypeptides disclosed herein can be administered directly to lymphoid tissues. Alternatively, lymphoid tissue specific targeting can be achieved using lymphoid tissue-specific transcriptional regulatory elements (TREs) such as a B lymphocyte-, T lymphocyte-, or dendritic cell-specific TRE. Lymphoid tissue specific TREs are known in the art.

Nucleic acids may also be administered in vivo by viral means. Nucleic acid molecules encoding fusion proteins may be packaged into retrovirus vectors using packaging cell lines that produce replication-defective retroviruses, as is well-known in the art. Other virus vectors may also be used, including recombinant adenoviruses and vaccinia virus, which can be rendered non-replicating. In addition to naked DNA or RNA, or viral vectors, engineered bacteria may be used as vectors.

Nucleic acids may also be delivered by other carriers, including liposomes, polymeric micro- and nanoparticles and polycations such as asialoglycoprotein/polylysine.

In addition to virus- and carrier-mediated gene transfer in vivo, physical means well-known in the art can be used for direct transfer of DNA, including administration of plasmid DNA and particle-bombardment mediated gene transfer.

5. Small Molecules

The immunomodulatory agent can be a small molecule. Small molecules agonists and antagonists AKT3 are known in the art or can be identified using routine screening methods.

In some embodiments, screening assays can include random screening of large libraries of test compounds. Alternatively, the assays may be used to focus on particular classes of compounds suspected of modulating the level of AKT3. Assays can include determinations of AKT3 mediated signaling activity. Other assays can include determinations of nucleic acid transcription or translation, mRNA levels, mRNA stability, mRNA degradation, transcription rates, and translation rates.

D. Pharmaceutical Compositions

One embodiment provides formulations of and pharmaceutical compositions including the disclosed Akt3 activators or inhibitors. Generally, dosage levels, for the compounds disclosed herein are between about 0.0001 mg/kg of body weight to about 1,000 mg/kg, more preferably of 0.001 to 500 mg/kg, more preferably 0.01 to 50 mg/kg of body weight daily are administered to mammals.

Pharmaceutical compositions including the disclosed Akt3 modulators, with or without a delivery vehicle, are provided. Pharmaceutical compositions can be formulated for administration by parenteral (intramuscular, intraperitoneal, intravenous (IV), or subcutaneous injection), enteral, transmucosal (nasal, vaginal, rectal, or sublingual), or transdermal (either passively or using iontophoresis or electroporation) routes of administration or using bioerodible inserts and can be formulated in dosage forms appropriate for each route of administration.

In certain embodiments, the compositions are administered locally, for example by injection directly into a site to be treated (e.g., into a tumor). In some embodiments, the compositions are injected or otherwise administered directly into the vasculature onto vascular tissue at or adjacent to the intended site of treatment (e.g., adjacent to a tumor). Typically, local administration causes an increased localized concentration of the composition which is greater than that which can be achieved by systemic administration.

1. Formulations for Parenteral Administration

Compounds and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants, and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and re-dissolved/resuspended immediately before use. The formulation may be sterilized by, for example, filtration through a bacteria retaining filter, by incorporating sterilizing agents into the compositions, by irradiating the compositions, or by heating the compositions.

2. Enteral Formulations

Suitable oral dosage forms include tablets, capsules, solutions, suspensions, syrups, and lozenges. Tablets can be made using compression or molding techniques well known in the art. Gelatin or non-gelatin capsules can be prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants. Delayed release dosage formulations may be prepared as described in standard references. These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), zein, shellac, and polysaccharides.

Additionally, the coating material may contain conventional carriers such as plasticizers, pigments, colorants, glidants, stabilization agents, pore formers, and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate, and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose, and sorbitol), polyethylene glycol, waxes, natural and synthetic gums such as acacia, tragacanth, sodium alginate, and cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid, and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or crosslinked polymers, such as cross-linked PVP (Polyplasdone® XL from GAF Chemical Corp).

Stabilizers are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

Oral dosage forms, such as capsules, tablets, solutions, and suspensions, can be formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation into the finished dosage form.

In another embodiment, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more compounds, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the compounds and/or additional active agents.

Extended Release Dosage Forms

The extended release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tradename Eudragit®. In further preferred embodiments, the acrylic polymer comprises a mixture of two acrylic resin lacquers commercially available from Rohm Pharma under the tradenames Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. Edragit® S-100 and Eudragit® L-100 are also preferred. The code designations RL (high permeability) and RS (low permeability) refer to the permeability properties of these agents. Eudragit® RL/RS mixtures are insoluble in water and in digestive fluids. However, multiparticulate systems formed to include the same are swellable and permeable in aqueous solutions and digestive fluids.

The polymers described above such as Eudragit® RL/RS may be mixed together in any desired ratio in order to ultimately obtain a sustained-release formulation having a desirable dissolution profile. Desirable sustained release multiparticulate systems may be obtained, for instance, from 100% Eudragit® RL, 50% Eudragit® RL and 50% Eudragit® RS, and 10% Eudragit® RL and 90% Eudragit® RS. One skilled in the art will recognize that other acrylic polymers may also be used, such as, for example, Eudragit® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules, etc.

An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art, such as direct compression, wet granulation, or dry granulation processes. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride, and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose, and waxes can also serve as binders. A lubricant is necessary in a tablet formulation to prevent the tablet and punches from sticking in the die. The lubricant is chosen from such slippery solids as talc, magnesium and calcium stearate, stearic acid, and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragit® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads, and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil, and acetylated monoglycerides. A stabilizing agent is preferably used to stabilize particles in the dispersion. Typical stabilizing agents are nonionic emulsifiers such as sorbitan esters, polysorbates, and polyvinylpyrrolidone. Glidants are recommended to reduce sticking effects during film formation and drying and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

3. Formulations for Pulmonary and Mucosal Administration

Active agent(s) and compositions thereof can be applied formulated for pulmonary or mucosal administration. The administration can include delivery of the composition to the lungs, nasal, oral (sublingual, buccal), vaginal, or rectal mucosa.

In one embodiment, the compounds are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the sub epithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging, e.g., from about pH 4.0 to about pH 7.4 or, from about pH 6.0 to about pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, and PBS. Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl-p-hydroxybenzoate.

In another embodiment, solvents that are low toxicity organic (i.e., nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PART LC Jet+ nebulizer (PART Respiratory Equipment, Monterey, Calif.).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter, and tap density for localized delivery to selected regions of the respiratory tract such as the deep lung or upper airways. For example, higher density or larger particles may be used for upper airway delivery. Similarly, a mixture of different sized particles, provided with the same or different EGS may be administered to target different regions of the lung in one administration.

Formulations for pulmonary delivery include unilamellar phospholipid vesicles, liposomes, or lipoprotein particles. Formulations and methods of making such formulations containing nucleic acid are well known to one of ordinary skill in the art. Liposomes are formed from commercially available phospholipids supplied by a variety of vendors including Avanti Polar Lipids, Inc. (Birmingham, Ala.). In one embodiment, the liposome can include a ligand molecule specific for a receptor on the surface of the target cell to direct the liposome to the target cell.

4. Transdermal

Transdermal formulations may also be prepared. These will typically be ointments, lotions, sprays, or patches, all of which can be prepared using standard technology. Transdermal formulations can include penetration enhancers.

### IV. Combination Therapy

The disclosed Akt3 modulators can be administered to a subject in need thereof alone or in combination with one or more additional therapeutic agents. In some embodiments, the Akt3 modulators and the additional therapeutic agent are administered separately, but simultaneously. The Akt3 modulators and the additional therapeutic agent can also be administered as part of the same composition. In other embodiments, the Akt3 modulators and the second therapeutic agent are administered separately and at different times, but as part of the same treatment regime.

The subject can be administered a first therapeutic agent 1, 2, 3, 4, 5, 6, or more hours, or 1, 2, 3, 4, 5, 6, 7, or more days before administration of a second therapeutic agent. In some embodiments, the subject can be administered one or more doses of the first agent every 1, 2, 3, 4, 5, 6 7, 14, 21, 28, 35, or 48 days prior to a first administration of second agent. The Akt3 modulators can be the first or the second therapeutic agent.

The Akt3 modulators and the additional therapeutic agent can be administered as part of a therapeutic regimen. For example, if a first therapeutic agent can be administered to a subject every fourth day, the second therapeutic agent can be administered on the first, second, third, or fourth day, or combinations thereof. The first therapeutic agent or second therapeutic agent may be repeatedly administered throughout the entire treatment regimen.

Exemplary molecules include, but are not limited to, cytokines, chemotherapeutic agents, radionuclides, other immunotherapeutics, enzymes, antibiotics, antivirals (especially protease inhibitors alone or in combination with nucleosides for treatment of HIV or Hepatitis B or C), anti-parasites (helminths, protozoans), growth factors, growth inhibitors, hormones, hormone antagonists, antibodies and bioactive fragments thereof (including humanized, single chain, and chimeric antibodies), antigen and vaccine formulations (including adjuvants), peptide drugs, anti-inflammatories, ligands that bind to Toll-like receptors (including but not limited to CpG oligonucleotides) to activate the innate immune system, molecules that mobilize and optimize the adaptive immune system, other molecules that activate or up-regulate the action of cytotoxic T lymphocytes, NK cells and helper T-cells, and other molecules that deactivate or down-regulate suppressor or regulatory T-cells.

The additional therapeutic agents are selected based on the condition, disorder or disease to be treated. For example, the Akt3 modulators can be co-administered with one or more additional agents that function to enhance or promote an immune response or reduce or inhibit an immune response.

A. Chemotherapeutic Agents

The disclosed Akt3 modulators can be combined with one or more chemotherapeutic agents and pro-apoptotic agents. Representative chemotherapeutic agents include, but are not limited to, amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxycarbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. Representative pro-apoptotic agents include, but are not limited to fludarabinetaurosporine, cycloheximide, actinomycin D, lactosylceramide, 15d-PGJ(2), and combinations thereof.

B. Anti-Inflammatories

Other suitable therapeutic agents include, but are not limited to, anti-inflammatory agents. The anti-inflammatory agent can be non-steroidal, steroidal, or a combination thereof. One embodiment provides oral compositions containing about 1% (w/w) to about 5% (w/w), typically about 2.5% (w/w) or an anti-inflammatory agent. Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed.

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

C. Immunosuppressive Agents

In some embodiments, the compound disclosed herein decreases Treg activity or production. In some embodiments, the compound disclosed herein is used in induction therapy for cancer. In some embodiments, the compound disclosed herein is used in combination with other immune therapeutic agents, immune modulators, costimulatory activating agonists, other cytokines and chemokines and factors, vaccines, oncolytic viruses, cell therapy, small molecules and targeted therapy, chemotherapy and radiation therapy. In some embodiments, the immune modulators include check point inhibitors such as anti-PD1, anti-CTLA4, anti-TIM3, anti-LAG3. In some embodiments, the costimulatory activating agonists including anti-OX40, anti-GITR, and the like. In some embodiments, the cell therapy includes engineered T cells, CAR-T, TCR-Tcells and others.

In some embodiments, the compound disclosed herein is used in combination with other immune therapeutic agents, immune modulators, biologics (e.g., antibodies), vaccines, small molecules and targeted therapy, anti-inflammatory, cell therapy (e.g., engineered Tregs and other type of cells, chemotherapy and radiation therapy.

In some embodiments, the compound disclosed herein, either used alone or in combination with other agents, is administered in vivo to a patient by intravenous, intramuscular, or other parenteral means. They can also be administered by intranasal application, inhalation, rectally, vaginally, topically, orally, or as implants. In other embodiments, the compound disclosed herein, either used alone or in combination with other agents, is applied ex vivo to enhance the function of suppressive Tregs, including natural tregs, induce-Tregs, engineered Tregs and other type of suppressive T cells, which optionally can then be used to treat a patient.

In some embodiments, the additional therapeutic agent is an immune suppressant. Immunosuppressive agents include, but are not limited to antibodies against other lymphocyte surface markers (e.g., CD40, alpha-4 integrin) or against cytokines), fusion proteins (e.g., CTLA-4-Ig (Orencia®) TNFR-Ig (Enbrel®)), TNF-α blockers such as Enbrel, Remicade, Cimzia and Humira, cyclophosphamide (CTX) (i.e., Endoxan®, Cytoxan®, Neosar®, Procytox®, Revimmune™), methotrexate (MTX) (i.e., Rheumatrex®, Trexall®), belimumab (i.e., Benlysta®), or other immunosuppressive drugs (e.g., cyclosporin A, FK506-like compounds, rapamycin compounds, or steroids), anti-proliferatives, cytotoxic agents, or other compounds that may assist in immunosuppression.

In some embodiments, the additional therapeutic agent can be a checkpoint inhibitor. In some embodiments, the therapeutic agent can be a CTLA-4 fusion protein, such as CTLA-4-Ig (abatacept). CTLA-4-Ig fusion proteins compete with the co-stimulatory receptor, CD28, on T cells for binding to CD80/CD86 (B7-1/B7-2) on antigen presenting cells, and thus function to inhibit T cell activation. In another embodiment, the therapeutic agent is a CTLA-4-Ig fusion protein known as belatacept. Belatacept contains two amino acid substitutions (L104E and A29Y) that markedly increase its avidity to CD86 in vivo. In another embodiment, the therapeutic agent is Maxy-4.

In another embodiment, the therapeutic agent is cyclophosphamide (CTX). Cyclophosphamide (the generic name for Endoxan®, Cytoxan®, Neosan®, Procytox®, Revimmune™), also known as cytophosphane, is a nitrogen mustard alkylating agent from the oxazophorines group.

The therapeutic agent can be administered in an effective amount to reduce the blood or serum levels of anti-double stranded DNA (anti-ds DNA) auto antibodies and/or to reduce proteinuria in a patient in need thereof.

In another embodiment, the therapeutic agent increases the amount of adenosine in the serum, see, for example, WO 08/147482 (incorporated herein by reference in its entirety). For example, the second therapeutic agent can be CD73-Ig, recombinant CD73, or another agent (e.g., a cytokine or monoclonal antibody or small molecule) that increases the expression of CD73, see, for example WO 04/084933 (incorporated herein by reference in its entirety). In another embodiment the therapeutic agent is Interferon-beta.

The therapeutic agent can be a small molecule that inhibits or reduces differentiation, proliferation, activity, and/or cytokine production and/or secretion by Th1, Th17, Th22, and/or other cells that secrete, or cause other cells to secrete, inflammatory molecules, including, but not limited to, IL-1(3, TNF-α, TGF-beta, IFN-γ, IL-18 IL-17, IL-6, IL-23, IL-22, IL-21, and MMPs. In another embodiment, the therapeutic agent is a small molecule that interacts with Tregs, enhances Treg activity, promotes or enhances IL-10 secretion by Tregs, increases the number of Tregs, increases the suppressive capacity of Tregs, or combinations thereof.

In some embodiments, the composition increases Treg activity or production. Exemplary Treg enhancing agents include but are not limited to glucocorticoid fluticasone, salmeteroal, antibodies to IL-12, IFN-γ, and IL-4; vitamin D3, and dexamethasone, and combinations thereof.

In some embodiments, the therapeutic agent is an antibody, for example, a functions blocking antibody against a proinflammatory molecule such as IL-6, IL-23, IL-22, or IL-21.

As used herein the term “rapamycin compound” includes the neutral tricyclic compound rapamycin, rapamycin derivatives, rapamycin analogs, and other macrolide compounds which are thought to have the same mechanism of action as rapamycin (e.g., inhibition of cytokine function). The language “rapamycin compounds” includes compounds with structural similarity to rapamycin, e.g., compounds with a similar macrocyclic structure, which have been modified to enhance their therapeutic effectiveness. Exemplary rapamycin compounds are known in the art (see, e.g. WO95122972; WO 95116691; WO 95104738; U.S. Pat. Nos. 6,015,809; 5,989,591; 5,567,709; 5,559,112; 5,530,006; 5,484,790; 5,385,908; 5,202,332; 5,162,333; 5,780,462; 5,120,727; each incorporated herein by reference in its entirety).

The language “FK506-like compounds” includes FK506, and FK506 derivatives and analogs, e.g., compounds with structural similarity to FK506, e.g., compounds with a similar macrocyclic structure which have been modified to enhance their therapeutic effectiveness. Examples of FK506-like compounds include, for example, those described in WO 00101385 (incorporated herein by reference in its entirety). In some embodiments, the language “rapamycin compound” as used herein does not include FK506-like compounds.

D. Treatments for Neurodegenerative Diseases

The disclosed Akt3 modulators can be administered with a second therapeutic that is selected based on the subject's disease state. The second therapeutic can be a treatment for Alzheimer's disease. Current treatments for Alzheimer's disease include but are not limited to cholinesterase inhibitors such as donepezil, rivastigmine, and galantamine; memantine; antidepressants such as citalopram, fluoxetine, paroxetine, sertraline, and trazadone; anxiolytics such as lorazepam and oxazepam; and antipsychotics such as aripiprazole, clozapine, haloperidol, olanzapine, quetiapine, risperidone, and ziprasidone.

In another embodiment, the additional therapeutic agent can be a treatment for ALS. There are currently two U.S. FDA approved treatments for ALS, riluzole and edavarone. Both drugs have been shown to slow down the progression of ALS. In addition to riluzole and edavarone, subjects with ALS can also be treated with drugs that target a specific symptom of the disease. Exemplary drugs include but are not limited to drugs to reduce spasticity such as antispastics like baclofen, dantrolene, and diazepam; drugs to help control nerve pain such as amitriptyline, carbamazepine, duloxetine, gabapentin, lamotrigine, milnacipran, nortriptyline, pregabalin and venlafaxine; and drugs to help patients swallow such as trihexyphenidyl or amitriptyline.

In one embodiment, the additional therapeutic agent can be a treatment for Parkinson's disease. Current treatments for Parkinson's disease include but are not limited to carbidopa-levodopa; dopamine agonists such as pramipexole, ropinirole, and rotigotine; MAO B inhibitors such as selegiline, rasagiline, and safinamide; catechol O-methyltransferase inhibitors such as entacapone and tolcapone; anticholinergics such as bentztropine and trihexyphenidyl; and amantadine.

The second therapeutic agent can be a treatment for Huntington's disease. Current treatments for Huntington's disease include but are not limited to tetrabenazine; antipsychotics such as haloperidol, chlorpromazine, risperidone, and quetiapine; amantadine; levetiracetam; clonazepam; antidepressants such as citalopram, escitalopram, fluoxetine, and sertraline; and anticonvulsants such as valproate, carbamazepine, and lamotrigine.

E. Treatments for Weight Loss

In one embodiment, the disclosed Akt3 modulators can be administered to a subject with an additional therapeutic agent that is used to treat cachexia or extreme weight loss. The current strategy for treating cachexia and extreme weight loss is to improve appetite by using appetite stimulants to ensure adequate intake of nutrients. Pharmacological interventions with appetite stimulants, nutrient supplementation, 5-HT3 antagonists and Cox-2 inhibitor have been used to treat cachexia.

In one embodiment, appetite stimulants are vitamins, minerals, or herbs including but not limited to zinc, thiamine, or fish oil. In another embodiment, the appetite stimulant is a medication including but not limited to dronabinol, megesterol, and oxandrolone.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

1. A method of treating a disease in a subject in need thereof comprising administering to the subject a composition comprising an Akt3 modulator in an amount effective to modulate Akt3 signaling and treat or delay the progression of the disease.

2. The method of claim 1, wherein the disease is selected from the group consisting of neurodegenerative disease, cachexia, anorexia, obesity's complication, inflammatory disease, viral-induced inflammatory reaction, Gulf War Syndrome, tuberous sclerosis, retinitis pigmentosa, transplant rejection, cancer, ischemic tissue injury, traumatic tissue injury, and a combination thereof.

3. The method of claim 2, wherein the disease is neurodegenerative disease.

4. The method of claim 3, wherein the neurodegenerative disease is selected from the group consisting of Parkinson's disease, Alzheimer's disease, amyotrophic lateral sclerosis, Motor Neuron Disease, Huntington's disease, HIV-induced neurodegeneration, Lewy Body Disease, spinal muscular atrophy, prion disease, spinocerebellar ataxia, familial amyloid polyneuropathy, and a combination thereof.

5. The method of claim 2, wherein the disease is cachexia or anorexia.

6. The method of claim 2, wherein the disease is obesity's complication.

7. The method of claim 6, wherein the obesity's complication is selected from the group consisting of glucose intolerance, hepatic steatosis, dyslipidemia, and a combination thereof.

8. The method of claim 2, wherein the disease is inflammatory disease.

9. The method of claim 8, wherein the inflammatory disease is selected from the group consisting of atopic dermatitis, allergy, asthma, and a combination thereof.

10. The method of claim 2, wherein the disease is viral-induced inflammatory reaction.

11. The method of claim 10, wherein the viral-induced inflammatory reaction is SARS-induced inflammatory pneumonitis, coronavirus disease 2019, or a combination thereof.

12. The method of claim 2, wherein the disease is Gulf War Syndrome or tuberous sclerosis.

13. The method of claim 2, wherein the disease is retinitis pigmentosa or transplant rejection.

14. The method of claim 2, wherein the disease is ischemic tissue injury or traumatic tissue injury.

15. The method of claim 2, wherein the disease is cancer.

16. The method of claim 15, wherein the cancer is selected from the group consisting of adult T-cell leukemia/lymphoma, bladder, brain, breast, cervical, colorectal, esophageal, kidney, liver, lung, nasopharyngeal, pancreatic, prostate, skin, stomach, uterine, ovarian, and testicular cancer.

17. The method of claim 15, wherein the cancer is leukemia.

18. The method of claim 17, wherein the leukemia is adult T-cell leukemia/lymphoma.

19. The method of claim 18, wherein the adult T-cell leukemia/lymphoma is caused by human T-cell lymphotropic virus.

20. The method of claim 1, wherein Akt3 is modulated in immune cells.

21. The method of claim 20, wherein the immune cells are selected from the group consisting of T cells, B cells, macrophages, and glial cells.

22. The method of claim 21, wherein the glial cells are astrocytes, microglia, or oligodendrocytes.

23. The method of claim 21, wherein the T cells are T regulatory cells.

24. The method of claim 1, wherein the Akt3 modulator activates Akt3 signaling.

25. The method of claim 1, wherein the Akt3 modulator inhibits Akt3 signaling.

26. The method of claim 1, wherein the Akt3 modulator increases T regulatory cell activity or production.

27. The method of claim 1, wherein the Akt3 modulator decreases T regulatory cell activity or production.

28. The method of claim 1, wherein the Akt3 modulator is a compound according to Formula I: 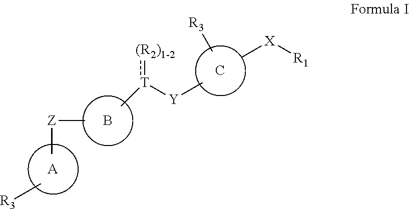
or a pharmaceutically acceptable enantiomer, salt, or solvate thereof, wherein:
rings A, B, and C are independently six-membered aryl or N-containing heteroaryl mono- or bicyclic ring systems containing zero or more N-atoms selected from the group consisting of phenyl, pyridine, pyrimidine, pyridazine, pyrazine, triazine, quinoline, quinazoline, isoquinoline, naphthalene, naphthyridine, indole, isoindole, cinnoline, phthalazine, quinoxaline, pteridine, purine, and benzimidazole;
R₁ is —(C₁-C₃₀)-alkyl, —(C₃-C₁₂)-cycloalkyl, —(C₃-C₁₂)-heterocycloalkyl, —(C₆-C₂₀)-aryl, or —(C₃-C₂₀)-heteroaryl groups optionally substituted by one or more substituents selected from —(C₁-C₁₂)-alkyl, —(C₃-C₁₂)-cycloalkyl, —(C₃-C₁₂)-heterocycloalkyl, —O—(C₁-C₁₂)-alkyl, —O—(C₁-C₁₂)-alkyl-(C₆-C₂₀)-aryl, —O—(C₃-C₁₂)-cycloalkyl, —S—(C₁-C₁₂)-alkyl, —S—(C₃-C₁₂)-cycloalkyl, —COO—(C₁-C₁₂)-alkyl, —COO—(C₃-C₁₂)-cycloalkyl, —CONH—(C₁-C₁₂)-alkyl, —CONH—(C₃-C₁₂)-cycloalkyl, —CO—(C₁-C₁₂)-alkyl, —CO—(C₃-C₁₂)-cycloalkyl, —N—[(C₁-C₁₂)-alkyl]₂, —(C₆-C₂₀)-aryl, —(C₆-C₂₀)-aryl-(C₁-C₁₂)-alkyl, —(C₆-C₂₀)-aryl-O—(C₁-C₁₂)-alkyl, —(C₃-C₂₀)-heteroaryl, —(C₃-C₂₀)-heteroaryl-(C₁-C₁₂)-alkyl, —(C₃-C₂₀)-heteroaryl-O—(C₁-C₁₂)-alkyl, —COOH, —OH, —SH, —SO₃H, —CN, —NH₂, or a halogen;
X, Y, and Z are independently ═O, —NH, —S, —N—(C₁-C₃₀)-alkyl, or —(C₁-C₃₀)-aryl; 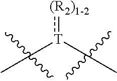
is —CH((C₁-C₃₀)-alkyl)), —(C═O)—, —CH(OH), —SO₂—, —SO—, or —CH(SOCH₃)—; and
R₃ is —(C₁-C₃₀)-alkyl, —(C₃-C₁₂)-cycloalkyl, —(C₃-C₁₂)-heterocycloalkyl, —(C₆-C₂₀)-aryl, or —(C₃-C₂₀)-heteroaryl groups optionally substituted by one or more substituents selected from —(C₁-C₁₂)-alkyl, —(C₃-C₁₂)-cycloalkyl, —(C₃-C₁₂)-heterocycloalkyl, —O—(C₁-C₁₂)-alkyl, —O—(C₁-C₁₂)-alkyl-(C₆-C₂₀)-aryl, —O—(C₃-C₁₂)-cycloalkyl, —S—(C₁-C₁₂)-alkyl, —S—(C₃-C₁₂)-cycloalkyl, —COO—(C₁-C₁₂)-alkyl, —COO—(C₃-C₁₂)-cycloalkyl, —CONH—(C₁-C₁₂)-alkyl, —CONH—(C₃-C₁₂)-cycloalkyl, —CO—(C₁-C₁₂)-alkyl, —CO—(C₃-C₁₂)-cycloalkyl, —N—[(C₁-C₁₂)-alkyl]₂, —(C₆-C₂₀)-aryl, —(C₆-C₂₀)-aryl-(C₁-C₁₂)-alkyl, —(C₆-C₂₀)-aryl-O—(C₁-C₁₂)-alkyl, —(C₃-C₂₀)-heteroaryl, —(C₃-C₂₀)-heteroaryl-(C₁-C₁₂)-alkyl, —(C₃-C₂₀)-heteroaryl-O—(C₁-C₁₂)-alkyl, —COOH, —OH, —SH, —SO₃H, —CN, —NH₂, or a halogen.

29. The method of claim 28, wherein the Akt3 modulator is a compound according to Formula II: 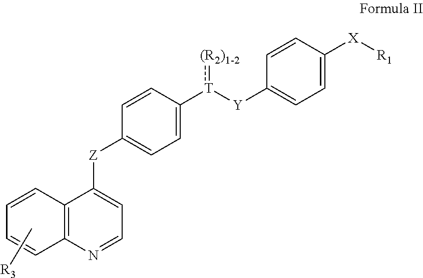
or a pharmaceutically acceptable enantiomer, salt, or solvate thereof, wherein:
R₁ is —(C₁-C₃₀)-alkyl, —(C₃-C₁₂)-cycloalkyl, —(C₃-C₁₂)-heterocycloalkyl, —(C₆-C₂₀)-aryl, or —(C₃-C₂₀)-heteroaryl groups optionally substituted by one or more substituents selected from —(C₁-C₁₂)-alkyl, —(C₃-C₁₂)-cycloalkyl, —(C₃-C₁₂)-heterocycloalkyl, —O—(C₁-C₁₂)-alkyl, —O—(C₁-C₁₂)-alkyl-(C₆-C₂₀)-aryl, —O—(C₃-C₁₂)-cycloalkyl, —S—(C₁-C₁₂)-alkyl, —S—(C₃-C₁₂)-cycloalkyl, —COO—(C₁-C₁₂)-alkyl, —COO—(C₃-C₁₂)-cycloalkyl, —CONH—(C₁-C₁₂)-alkyl, —CONH—(C₃-C₁₂)-cycloalkyl, —CO—(C₁-C₁₂)-alkyl, —CO—(C₃-C₁₂)-cycloalkyl, —N—[(C₁-C₁₂)-alkyl]₂, —(C₆-C₂₀)-aryl, —(C₆-C₂₀)-aryl-(C₁-C₁₂)-alkyl, —(C₆-C₂₀)-aryl-O—(C₁-C₁₂)-alkyl, —(C₃-C₂₀)-heteroaryl, —(C₃-C₂₀)-heteroaryl-(C₁-C₁₂)-alkyl, —(C₃-C₂₀)-heteroaryl-O—(C₁-C₁₂)-alkyl, —COOH, —OH, —SH, —SO₃H, —CN, —NH₂, or a halogen;
X, Y, and Z are independently —O, —NH, —S, —N—(C₁-C₃₀)-alkyl, or —(C₁-C₃₀)-aryl; 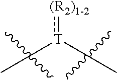
is —CH((C₁-C₃₀)-alkyl))—, —(C═O)—, —CH(OH), —SO₂—, —SO—, or —CH(SOCH₃)—; and
R₃ is —(C₁-C₃₀)-alkyl, —(C₃-C₁₂)-cycloalkyl, —(C₃-C₁₂)-heterocycloalkyl, —(C₆-C₂₀)-aryl, or —(C₃-C₂₀)-heteroaryl groups optionally substituted by one or more substituents selected from —(C₁-C₁₂)-alkyl, —(C₃-C₁₂)-cycloalkyl, —(C₃-C₁₂)-heterocycloalkyl, —O—(C₁-C₁₂)-alkyl, —O—(C₁-C₁₂)-alkyl-(C₆-C₂₀)-aryl, —O—(C₃-C₁₂)-cycloalkyl, —S—(C₁-C₁₂)-alkyl, —S—(C₃-C₁₂)-cycloalkyl, —COO—(C₁-C₁₂)-alkyl, —COO—(C₃-C₁₂)-cycloalkyl, —CONH—(C₁-C₁₂)-alkyl, —CONH—(C₃-C₁₂)-cycloalkyl, —CO—(C₁-C₁₂)-alkyl, —CO—(C₃-C₁₂)-cycloalkyl, —N—[(C₁-C₁₂)-alkyl]₂, —(C₆-C₂₀)-aryl, —(C₆-C₂₀)-aryl-(C₁-C₁₂)-alkyl, —(C₆-C₂₀)-aryl-O—(C₁-C₁₂)-alkyl, —(C₃-C₂₀)-heteroaryl, —(C₃-C₂₀)-heteroaryl-(C₁-C₁₂)-alkyl, —(C₃-C₂₀)-heteroaryl-O—(C₁-C₁₂)-alkyl, —COOH, —OH, —SH, —SO₃H, —CN, —NH₂, or a halogen.

30. The method of claim 28, wherein the Akt3 modulator is a compound according to Formula III: 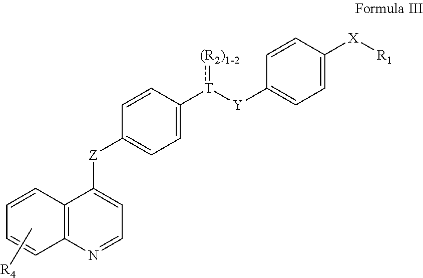
or a pharmaceutically acceptable enantiomer, salt, or solvate thereof, wherein:
R₁ is —(C₁-C₃₀)-alkyl, —(C₃-C₁₂)-cycloalkyl, —(C₃-C₁₂)-heterocycloalkyl, —(C₆-C₂₀)-aryl, or —(C₃-C₂₀)-heteroaryl groups optionally substituted by one or more substituents selected from —(C₁-C₁₂)-alkyl, —(C₃-C₁₂)-cycloalkyl, —(C₃-C₁₂)-heterocycloalkyl, —O—(C₁-C₁₂)-alkyl, —O—(C₁-C₁₂)-alkyl-(C₆-C₂₀)-aryl, —O—(C₃-C₁₂)-cycloalkyl, —S—(C₁-C₁₂)-alkyl, —S—(C₃-C₁₂)-cycloalkyl, —COO—(C₁-C₁₂)-alkyl, —COO—(C₃-C₁₂)-cycloalkyl, —CONH—(C₁-C₁₂)-alkyl, —CONH—(C₃-C₁₂)-cycloalkyl, —CO—(C₁-C₁₂)-alkyl, —CO—(C₃-C₁₂)-cycloalkyl, —N—[(C₁-C₁₂)-alkyl]₂, —(C₆-C₂₀)-aryl, —(C₆-C₂₀)-aryl-(C₁-C₁₂)-alkyl, —(C₆-C₂₀)-aryl-O—(C₁-C₁₂)-alkyl, —(C₃-C₂₀)-heteroaryl, —(C₃-C₂₀)-heteroaryl-(C₁-C₁₂)-alkyl, —(C₃-C₂₀)-heteroaryl-O—(C₁-C₁₂)-alkyl, —COOH, —OH, —SH, —SO₃H, —CN, —NH₂, or a halogen;
X, Y, and Z are independently —O, —NH, —S, —N—(C₁-C₃₀)-alkyl, or —(C₁-C₃₀)-aryl; 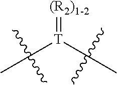
is —CH((C₁-C₃₀)-alkyl))—, —(C═O)—, —CH(OH), —SO₂—, —SO—, or —CH(SOCH₃)—; and
R₄ is —(C₁-C₁₂)-alkyl, —(C₃-C₁₂)-cycloalkyl, —(C₃-C₁₂)-heterocycloalkyl, —O—(C₁-C₁₂)-alkyl, —O—(C₁-C₁₂)-alkyl-(C₆-C₂₀)-aryl, —O—(C₃-C₁₂)-cycloalkyl, —S—(C₁-C₁₂)-alkyl, —S—(C₃-C₁₂)-cycloalkyl, —COO—(C₁-C₁₂)-alkyl, —COO—(C₃-C₁₂)-cycloalkyl, —CONH—(C₁-C₁₂)-alkyl, —CONH—(C₃-C₁₂)-cycloalkyl, —CO—(C₁-C₁₂)-alkyl, —CO—(C₃-C₁₂)-cycloalkyl, —N—[(C₁-C₁₂)-alkyl]₂, —(C₆-C₂₀)-aryl, —(C₆-C₂₀)-aryl-(C₁-C₁₂)-alkyl, —(C₆-C₂₀)-aryl-O—(C₁-C₁₂)-alkyl, —(C₃-C₂₀)-heteroaryl, —(C₃-C₂₀)-heteroaryl-(C₁-C₁₂)-alkyl, —(C₃-C₂₀)-heteroaryl-O—(C₁-C₁₂)-alkyl, —COOH, —OH, —SH, —SO₃H, —CN, —NH₂, or a halogen.

31. The method of claim 28, wherein the Akt3 modulator is a compound according to Formula IV: 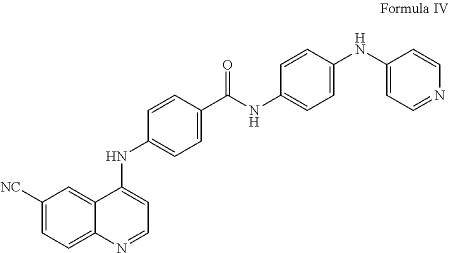 [SMILES: N#Cc1ccc2nccc(Nc3ccc(C(=O)Nc4ccc(Nc5ccncc5)cc4)cc3)c2c1]
or a pharmaceutically acceptable enantiomer, salt, or solvate thereof.

32. The method of claim 1, further comprising administering a second therapeutic agent to the subject.

33. The method of claim 32, wherein the second therapeutic agent is selected from the group consisting of a nutrient supplementation, a chemotherapeutic, an anti-inflammatory, an immunosuppressant, a cholinesterase inhibitor, an antidepressant, an anxiolytic, an antipsychotic, riluzole, edavarone, a dopamine agonist, a MAO B inhibitor, a catechol O-methyltransferase inhibitor, an anticholinergic, an anticonvulsant, tetrabenazine, carbidopa-levodopa, an antispastic, an antibody, a fusion protein, an enzyme, a nucleic acid, a ribonucleic acid, an anti-proliferative, a cytotoxic agent, an appetite stimulant, a 5-HT3 antagonist, a Cox-2 inhibitor, and a combination thereof.

34. A method of treating cachexia in a subject in need thereof comprising administering a composition comprising a selective inhibitor of Akt3 to the subject in an amount effective to inhibit Akt3 signaling in adipocytes and activate adipogenesis.

35. The method of claim 34, further comprising administering a second therapeutic agent to the subject.

36. The method of claim 35, wherein the second therapeutic agent is selected from the group consisting of an appetite stimulant, a nutrient supplementation, a 5-HT3 antagonist, a Cox-2 inhibitor, a chemotherapeutic, an anti-inflammatory, an immunosuppressant, a cholinesterase inhibitor, an antidepressant, an anxiolytic, an antipsychotic, riluzole, edavarone, a dopamine agonist, a MAO B inhibitor, a catechol O-methyltransferase inhibitor, an anticholinergic, an anticonvulsant, tetrabenazine, carbidopa-levodopa, an antispastic, an antibody, a fusion protein, an enzyme, a nucleic acid, a ribonucleic acid, an anti-proliferative, a cytotoxic agent, and a combination thereof.

37. The method of claim 36, wherein the second therapeutic agent is an appetite stimulant, a nutrient supplementation, a 5-HT3 antagonist, or a Cox-2 inhibitor.

38. The method of claim 34, wherein the subject has neurodegenerative disease, cachexia, anorexia, obesity's complication, inflammatory disease, viral-induced inflammatory reaction, Gulf War Syndrome, tuberous sclerosis, retinitis pigmentosa, transplant rejection, cancer, and a combination thereof.

39. The method of claim 1, wherein the Akt3 modulator is a compound selected from the group consisting of:

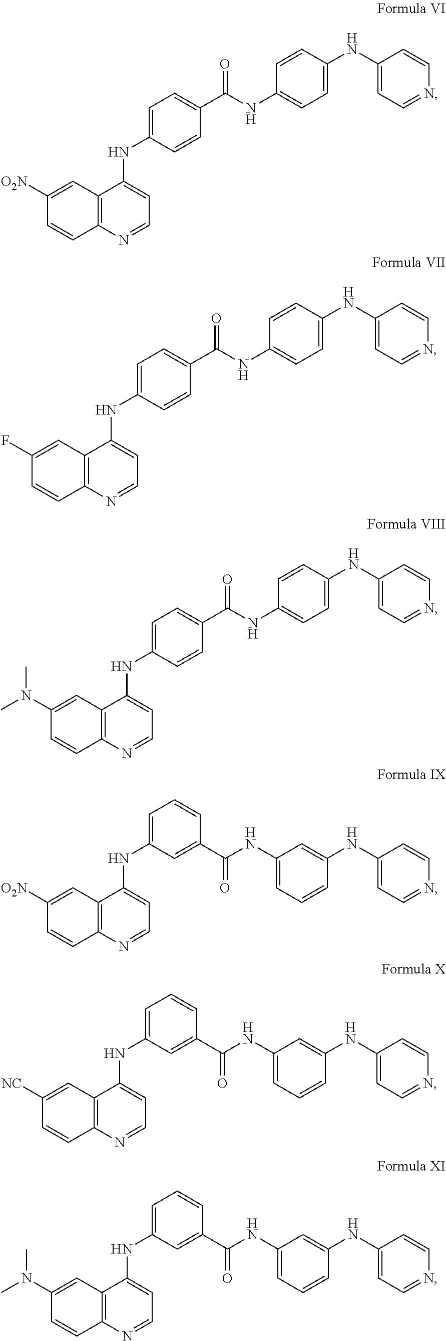 [SMILES: O=C(Nc1ccc(Nc2ccncc2)cc1)c1ccc(Nc2ccnc3ccc([N+](=O)[O-])cc23)cc1 ; O=C(Nc1ccc(Nc2ccncc2)cc1)c1ccc(Nc2ccnc3ccc(F)cc23)cc1 ; CN(C)c1ccc2nccc(Nc3ccc(C(=O)Nc4ccc(Nc5ccncc5)cc4)cc3)c2c1 ; O=C(Nc1cccc(Nc2ccncc2)c1)c1cccc(Nc2ccnc3ccc([N+](=O)[O-])cc23)c1 ; N#Cc1ccc2nccc(Nc3cccc(C(=O)Nc4cccc(Nc5ccncc5)c4)c3)c2c1 ; CN(C)c1ccc2nccc(Nc3cccc(C(=O)Nc4cccc(Nc5ccncc5)c4)c3)c2c1]

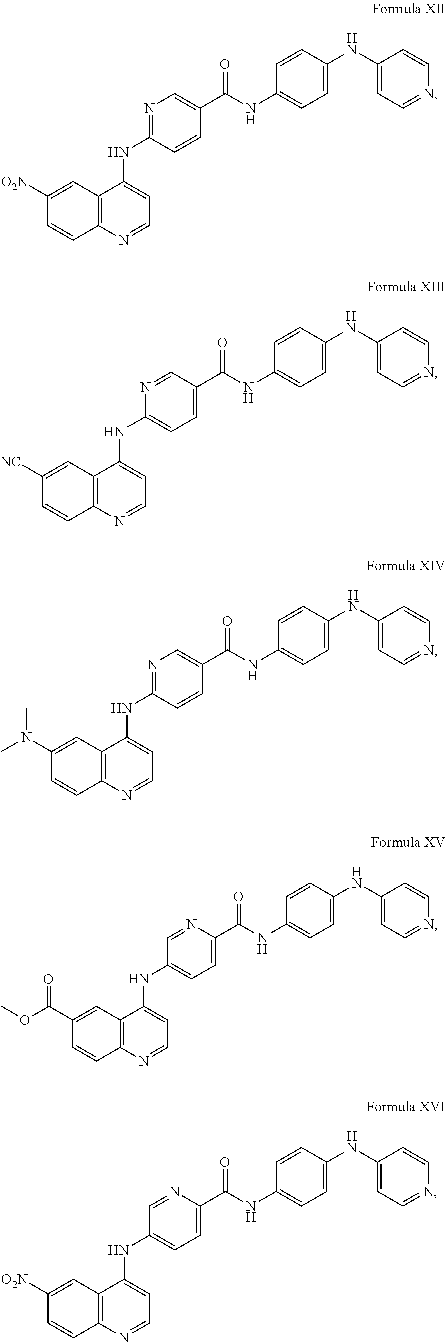 [SMILES: O=C(Nc1ccc(Nc2ccncc2)cc1)c1ccc(Nc2ccnc3ccc([N+](=O)[O-])cc23)nc1 ; N#Cc1ccc2nccc(Nc3ccc(C(=O)Nc4ccc(Nc5ccncc5)cc4)cn3)c2c1 ; CN(C)c1ccc2nccc(Nc3ccc(C(=O)Nc4ccc(Nc5ccncc5)cc4)cn3)c2c1 ; COC(=O)c1ccc2nccc(Nc3ccc(C(=O)Nc4ccc(Nc5ccncc5)cc4)nc3)c2c1 ; O=C(Nc1ccc(Nc2ccncc2)cc1)c1ccc(Nc2ccnc3ccc([N+](=O)[O-])cc23)cn1]

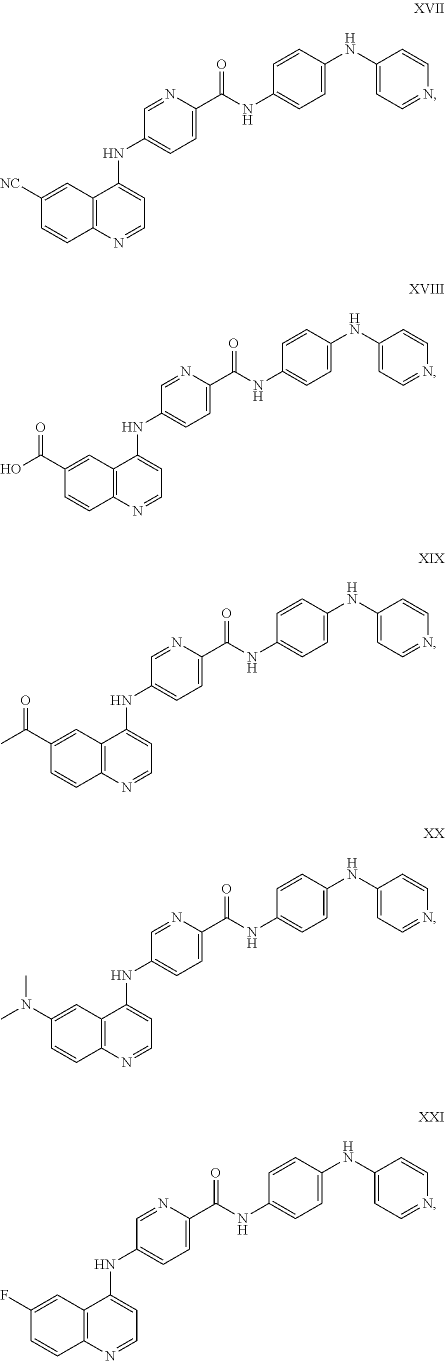 [SMILES: N#Cc1ccc2nccc(Nc3ccc(C(=O)Nc4ccc(Nc5ccncc5)cc4)nc3)c2c1 ; O=C(O)c1ccc2nccc(Nc3ccc(C(=O)Nc4ccc(Nc5ccncc5)cc4)nc3)c2c1 ; CC(=O)c1ccc2nccc(Nc3ccc(C(=O)Nc4ccc(Nc5ccncc5)cc4)nc3)c2c1 ; CN(C)c1ccc2nccc(Nc3ccc(C(=O)Nc4ccc(Nc5ccncc5)cc4)nc3)c2c1 ; O=C(Nc1ccc(Nc2ccncc2)cc1)c1ccc(Nc2ccnc3ccc(F)cc23)cn1]

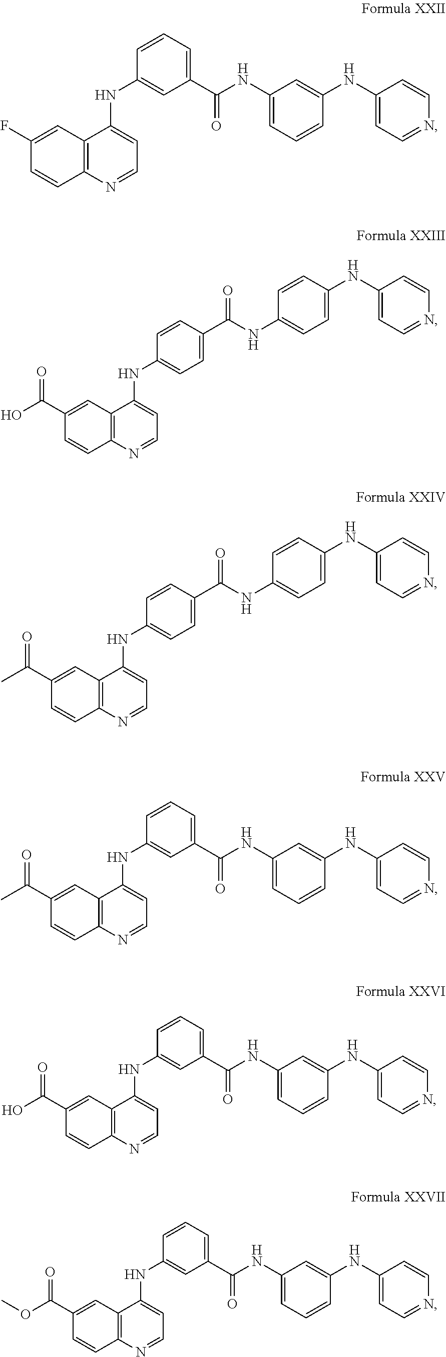 [SMILES: O=C(Nc1cccc(Nc2ccncc2)c1)c1cccc(Nc2ccnc3ccc(F)cc23)c1 ; O=C(O)c1ccc2nccc(Nc3ccc(C(=O)Nc4ccc(Nc5ccncc5)cc4)cc3)c2c1 ; CC(=O)c1ccc2nccc(Nc3ccc(C(=O)Nc4ccc(Nc5ccncc5)cc4)cc3)c2c1 ; CC(=O)c1ccc2nccc(Nc3cccc(C(=O)Nc4cccc(Nc5ccncc5)c4)c3)c2c1 ; O=C(O)c1ccc2nccc(Nc3cccc(C(=O)Nc4cccc(Nc5ccncc5)c4)c3)c2c1 ; COC(=O)c1ccc2nccc(Nc3cccc(C(=O)Nc4cccc(Nc5ccncc5)c4)c3)c2c1]

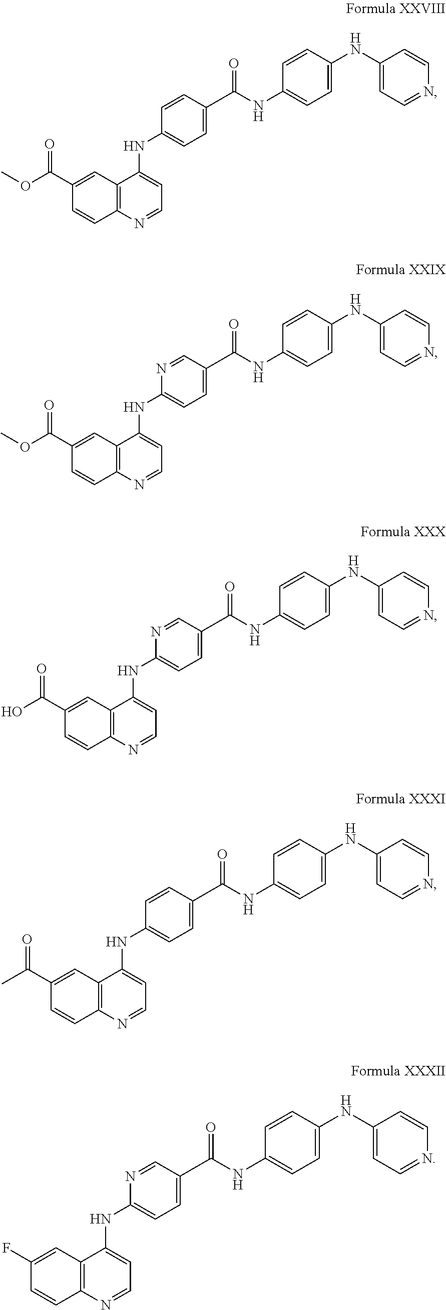 [SMILES: COC(=O)c1ccc2nccc(Nc3ccc(C(=O)Nc4ccc(Nc5ccncc5)cc4)cc3)c2c1 ; COC(=O)c1ccc2nccc(Nc3ccc(C(=O)Nc4ccc(Nc5ccncc5)cc4)cn3)c2c1 ; O=C(O)c1ccc2nccc(Nc3ccc(C(=O)Nc4ccc(Nc5ccncc5)cc4)cn3)c2c1 ; CC(=O)c1ccc2nccc(Nc3ccc(C(=O)Nc4ccc(Nc5ccncc5)cc4)cc3)c2c1 ; O=C(Nc1ccc(Nc2ccncc2)cc1)c1ccc(Nc2ccnc3ccc(F)cc23)nc1]

40. The method of claim 34, wherein the selective inhibitor of Akt3 is a compound selected from the group consisting of:
